(19) United States
(12) Patent Application Publication
(10) Pub. No.: US 2025/0329409 A1
(43) Pub. Date: Oct. 23, 2025

(54) GREEDY APPROACH TO IDENTIFYING PEPTIDES WITH MULTIPLE POST-TRANSLATIONAL MODIFICATIONS

(71) Applicant: The Hong Kong University of Science and Technology, Kowloon (HK)

(72) Inventors: Weichuan YU, Kowloon (HK); Ning LI, Clear Water Bay (HK); Shengzhi LAI, Kowloon (HK)

(21) Appl. No.: 19/092,512

(22) Filed: Mar. 27, 2025

Related U.S. Application Data
(60) Provisional application No. 63/635,630, filed on Apr. 18, 2024.

(51) Int. Cl. G16B 15/20; G16B 40/10

(52) U.S. Cl. CPC G16B 15/20; G16B 40/10

(57) ABSTRACT

A greedy approach to identifying peptides with multiple post-translational modification is provided. A method includes extracting tags from input data and reducing information indicative of a protein database. The extracting includes converting peaks in tandem mass spectra of the input data into a weighted directed graph, resulting in extracted tags. The tags represent sequential amino acids. The reducing includes determining respective coverages of proteins in the protein database using the extracted tags. Further, the method includes locating a selected tag in an indexed database configured for protein candidate retrieval and scoring ones of the proteins that comprise the selected tag. The selected tag is selected from the extracted tags. Further, the method includes using a greedy approach process that characterizes post-translational modification patterns of the selected tag based on the scoring. The method also includes, based on a result of the greedy approach process, implementing a quality control process.

### CROSS-REFERENCE TO RELATED APPLICATIONS

This application claims the benefit of priority to U.S. Provisional Application No. 63/635,630, filed Apr. 18, 2024, and entitled “A GREEDY APPROACH TO IDENTIFYING PEPTIDES WITH MULTIPLE POST-TRANSLATIONAL MODIFICATION,” the entirety of which is expressly incorporated herein by reference.

### BACKGROUND

Post-translational modifications (PTMs) are important in regulating cellular activities. Database search methods have been developed to identify peptides with PTMs and characterize PTM patterns. However, it still remains challenging to identify peptides with PTMs, especially peptides with multiple PTMs. Conventional methods have been facing the challenge of exponentially increasing the number of PTM combinations when identifying peptide with more than two PTMs. Accordingly, unique challenges related to database search methods exist and in view of peptides with multiple PTMs.

The above-described context with respect to database search methods is merely intended to provide an overview of current technology and is not intended to be exhaustive. Other contextual descriptions, and corresponding benefits of some of the various non-limiting embodiments described herein, will become further apparent upon review of the following detailed description.

### SUMMARY

The following presents a simplified summary of the disclosed subject matter to provide a basic understanding of some aspects of the various embodiments. This summary is not an extensive overview of the various embodiments. It is intended neither to identify key or critical elements of the various embodiments nor to delineate the scope of the various embodiments. Its sole purpose is to present some concepts of the disclosure in a streamlined form as a prelude to the more detailed description that is presented later.

An embodiment relates to a method that includes extracting, by a computing system comprising at least one processor, tags from input data. The extracting comprises converting peaks in tandem mass spectra of the input data into a weighted directed graph, resulting in extracted tags. The tags represent sequential amino acids. The method also includes reducing, by the computing system, information indicative of a protein database. The reducing comprises determining respective coverages of proteins in the protein database using the extracted tags. Further, the method includes locating, by the computing system, a selected tag in an indexed database configured for protein candidate retrieval. The selected tag is selected from the extracted tags. The method also includes scoring, by the computing system, ones of the proteins that comprise the selected tag. Further, the method includes using, by the computing system, a greedy approach process that characterizes post-translational modification patterns of the selected tag based on the scoring. The method also includes, based on a result of the greedy approach process, implementing, by the computing system, a quality control process based on the target-decoy strategy.

In an example, the tags comprise respective N-sections and respective C-sections. Further to this example, the using of the greedy approach process comprises using the greedy approach process on the respective N-sections and the respective C-sections.

Prior to the extracting, in some implementations, the method can include obtaining, by the computing system, the tandem mass spectra from a group of proteins with post-translational modifications. Further to these implementations, the method can include facilitating, by the computing system, digestion of samples of the group of proteins into peptides by an enzyme before using the tandem mass spectra. The method can also include transmitting, by the computing system, the samples with the post-translational modifications to a tandem mass spectrometer.

According to some implementations, the method can include determining, by the computing system, nodes and edges of a weighted directed graph that is used to extract tags. The determining of the nodes and the edges can include using peaks in the tandem mass spectra and potential amino acids that are located between peak pairs of the tags.

In an example, the extracting can include extracting the tags using a depth-first search process. In another example, the reducing can include removing any of the proteins determined to have a coverage level that is below a threshold coverage level. In yet another example, the locating can include using tags comprising ammino acid lengths between 3 amino acids and 9 amino acids for retrieval of protein candidates.

In accordance with some implementations, the method can include, prior to the locating, constructing, by the computing system, the indexed database that facilitates protein candidate retrieval using tags of various lengths. Further to these implementations, the constructing can include, based on a determination that the quality control process has been applied, generating a target indexed database and a decoy indexed database. The generating of the decoy indexed database can include shuffling target protein sequences.

According to some implementations, the using of the greedy approach process comprises iteratively including a current best post-translational modification pattern of the post-translational modification patterns with which a largest number of experimental peaks are matched to theoretical peaks. The experimental peaks are generated from the tandem mass spectra and the theoretical peaks are generated from protein candidates in the indexed database.

Another embodiment relates to a system that includes at least one processor and at least one memory that stores executable instructions that, when executed by the at least one processor, facilitate performance of operations. The operations can include extracting tags that represent sequential amino acids based on peaks in tandem mass spectra being converted into a weighted directed graph, resulting in extracted tags. The operations can also include reducing a protein database based on the extracted tags. The reducing can include removing, from the protein database, extracted tags determined to have a reliability level that is below a reliability threshold level. The reliability level can be based on a determination of respective coverages of proteins in the protein database. Further, the operations can include locating a tag in an indexed database, resulting in a located tag and scoring ones of the proteins that comprise the located tag. The extracted tags include the located tag. Based on the scoring, the operations can include characterizing post-translational modification patterns of the located tag. The characterizing comprises using a greedy approach process. Further, the operations can include, based on a result of the greedy approach process, implementing a quality control process.

According to an implementation, the operations can include, prior to the extracting, obtaining the tandem mass spectra from a group of proteins with post-translational modifications. In some implementations, the extracted tags can include tags of different lengths. Further to these implementations, the identifying is performed without user specification. In accordance with some implementations, the operations can include, prior to the identifying, generating the indexed database. Additionally, the indexed database can facilitate protein candidate retrieval using tags of different lengths.

Yet another embodiment relates to a computing system comprising at least one processor configured to retrieve peptide backbone candidates using tags of various lengths. The peptide backbone candidates comprise multiple post-translational modification patterns. The tags represent sequential amino acids. The at least one processor can also be configured to characterize post-translational modification patterns of the multiple post-translational modification patterns of the peptide backbone candidates by employing a greedy approach that simplifies a combinatorial problem into a linear problem, resulting in characterized candidates. Further, the at least one processor can be configured to score the characterized candidates in an indexed database, resulting in scored candidates, and apply a protein feedback process that re-ranks the scored candidates based on respective scores of proteins that contain the characterized candidates, resulting in re-ranked candidates. Further, the at least one processor can be configured to output the re-ranked candidates while concurrently controlling a false discovery rate with a quality control process.

According to an implementation, to characterize the post-translational modification patterns, the at least one processor is configured to identify peptides with multiple post-translational modifications in absence of any user specification. In some implementations, the quality control process facilitates estimation of an output quality applicable to the re-ranked candidates.

To the accomplishment of the foregoing and related ends, the disclosed subject matter includes one or more of the features hereinafter more fully described. The following description and the annexed drawings set forth in detail certain illustrative aspects of the subject matter. However, these aspects are indicative of but a few of the various ways in which the principles of the subject matter can be employed. Other aspects, advantages, and novel features of the disclosed subject matter will become apparent from the following detailed description when considered in conjunction with the drawings. It will also be appreciated that the detailed description can include additional or alternative embodiments beyond those described in this summary.

### BRIEF DESCRIPTION OF THE DRAWINGS

Various non-limiting embodiments are further described with reference to the accompanying drawings in which:

FIG. 1A illustrates an example, non-limiting, first stage of a system workflow in accordance with one or more embodiments described herein;

FIG. 1B illustrates an example, non-limiting, second stage of the system workflow in accordance with one or more embodiments described herein;

FIG. 1C illustrates an example, non-limiting, third stage of the system workflow in accordance with one or more embodiments described herein;

FIG. 2A illustrates a first chart showing performance results of various simulations performed in accordance with one or more embodiments described herein;

FIG. 2B illustrates a second chart showing performance results of precision of post-translational modification characterization of the various simulations performed in accordance with one or more embodiments described herein;

FIGS. 2C-2F illustrate subplots showing performance results of the sensitivity of post-translational modification characterization of the various simulations performed in accordance with one or more embodiments described herein;

FIG. 2G illustrates a legend that provides a guide to the colors, symbols, and lines used for FIGS. 2A through 2F in accordance with one or more embodiments described herein;

FIG. 3 illustrates a chart showing peptide length distribution of a data set according to one or more simulations performed in accordance with one or more embodiments described herein;

FIGS. 4A-4G illustrate subplots of the quality of peptide candidate list retrieved by tag-V or tag-ks for data sets H01 to H06, and Hmix, respectively, according to one or more simulations performed in accordance with one or more embodiments described herein;

FIG. 4H illustrates a histogram of the peptide length distribution for Hmix according to one or more simulations performed in accordance with one or more embodiments described herein;

FIG. 4I illustrates a legend that provides a guide to the colors, symbols, and/or lines used for FIGS. 4A through 4G in accordance with one or more embodiments described herein;

FIGS. 5A to 5E illustrate subplots of the quality of peptide candidate lists retrieved for data set SIMU according to one or more simulations performed in accordance with one or more embodiments described herein;

FIG. 5F illustrates a legend that provides a guide to the colors, symbols, and/or lines used for FIGS. 5A-5E in accordance with one or more embodiments described herein

FIG. 6 illustrates results of data sets F01 to F12 according to one or more simulations performed in accordance with one or more embodiments described herein;

FIG. 7 illustrates overall results of data set SIMU according to one or more simulations performed in accordance with one or more embodiments described herein;

FIG. 8 illustrates a chart of the true number of post-translational modifications in each tandem mass (MS2) spectrum and normalizing them as characterization rate according to one or more simulations performed in accordance with one or more embodiments described herein;

FIG. 9 illustrates an example, non-limiting, system for facilitating using a greedy approach to identify peptides with multiple post-translational modification in accordance with one or more embodiments described herein;

FIG. 10 illustrates an example, non-limiting, computer-implemented method that facilitates identification of peptides with multiple post-translational modification in accordance with one or more embodiments described herein;

FIG. 11 illustrates an example, non-limiting, computing environment in which one or more embodiments described herein can be facilitated; and

FIG. 12 illustrates an example, non-limiting, networking environment in which one or more embodiments described herein can be facilitated.

### DETAILED DESCRIPTION

One or more embodiments are now described more fully hereinafter with reference to the accompanying drawings in which example embodiments are shown. In the following description, for purposes of explanation, numerous specific details are set forth in order to provide a thorough understanding of the various embodiments. However, the various embodiments can be practiced without these specific details. In other instances, well-known structures and devices are shown in block diagram form in order to facilitate describing the various embodiments.

As discussed above, post-translational modifications (PTMs) are important in regulating cellular activities. Conventional database search methods have been developed to identify peptides with PTMs and characterize PTM patterns. However, it still remains challenging to identify peptides with PTMs, especially peptides with multiple PTMs. Conventional methods have been facing the challenge of exponentially increasing the number of PTM combinations when identifying peptide with more than two PTMs. Provided herein are embodiments related to a greedy approach that simplifies the PTM characterization problem into a linear problem, which enables characterizing multiple PTMs on one peptide. Also provided herein are details related to a comparison with conventional methods in order to illustrate the advantages of the disclosed embodiments.

In further detail, conventional methods for identifying peptides with PTMs suffer from a low sensitivity of backbone identification and a low precision of PTM characterization. This is mainly due to the large number of PTM combinations when considering multiple PTMs on peptides. Provided herein are embodiments (sometimes referred to as PIPI2) that have a high sensitivity in identifying peptides with multiple PTMs without user specification. The disclosed embodiments characterize PTMs with a greedy approach that simplifies the combinatorial problem into a linear problem, enabling it to manage peptides with multiple PTMs. Meanwhile, the disclosed embodiments combines tag of various lengths to increase the quality of peptide candidates. Compared to conventional methods, the disclosed embodiments show the highest precision and sensitivity in backbone identification and PTM characterization, especially for peptides with multiple PTMs. Moreover, when the data quality decreases, the embodiments provided herein are the only solution that maintains its performance. In real applications, the disclosed embodiments can identify many more peptides and depict the PTM profile of large-scale data sets as compared to conventional methods. Therefore, the disclosed embodiments provide more insight to the researchers in a PTM study.

Conventional database search using tandem mass (MS2) spectra has been widely used to identify peptides in bottom-up proteomics over the past three decades. Database search methods can identify peptides by calculating the similarity between experimental peaks in MS2 spectra and theoretical peaks of peptide candidate sequences. Among these methods, closed search retrieves peptide candidates from a protein database with a tight precursor mass tolerance, such as 10 parts per million (ppm). PTMs are biologically important features that regulate cellular functions. However, the presence of PTMs leads to at least two issues that decrease the identification rate of peptides: modifying the precursor mass and shifting the locations of peaks in MS2 spectra, which decrease the similarity measure in database search. The inconsistency between the precursor mass and the theoretical mass of the peptide backbone sequence (amino acid sequences only, disregarding PTMs) results in failures to retrieve the true sequence as a potential candidate and a false peptide-spectrum match (PSM).

One conventional technique bypassed the first issue by an open search with a large precursor mass tolerance (500 Daltons), which identified 46% more peptides than a closed search. Nevertheless, the correlation between an MS2 spectrum and the true peptide sequence can still be underestimated because of the shifted peaks. Some search engines allow for user-specified PTMs and append all modified peptide sequences to their database. However, the number of allowable user-specified PTMs is still limited because a large number of PTM combinations would increase the database size exponentially. Considering the importance of PTMs and the large number of PTM entries in a database (for example, a UNIMOD database, which is a public domain database of protein modifications) for mass spectrometry (MS) purposes, it is desirable to identify the backbone sequence and characterize the PTM patterns (e.g., the numbers, types, and sites of PTMs in peptides), without any prior user-specification.

Conventional methods that have been proposed to address the issue of identifying the backbone sequence and characterizing the PTM patterns without any prior user specification can be categorized into tag-based methods and non-tag-based methods. Tag-based methods start from extracting short sequential amino acids (tags) from MS2 spectra. Tags are locally unaffected by shifted peaks hence invariant to PTM. As for non-tag-based methods, the most representative one trades off storage for speed using a fragment-ion index and applies open search with precursor mass tolerance of 500 Daltons.

To overcome deficiencies of conventional methods as well as other issues, provided herein are various embodiments related to an analysis tool, referred to as PIPI2, that has high sensitivity in identifying peptides with multiple PTMs without user specification. The disclosed embodiments (PIPI2) first retrieve peptide backbone candidates using tags of various lengths and then characterize the PTM patterns with a greedy approach that simplifies the combinatorial problem into a linear problem, and finally apply a protein feedback module (PFM) to re-rank the scored candidates. With diverse data sets as provided herein, it has been demonstrated that the performance of the disclosed embodiments (PIPI2) on backbone identification and PTM characterization is much better than conventional analysis programs. Therefore, the disclosed embodiments are suitable for identifying peptides with multiple PTMs without user specification.

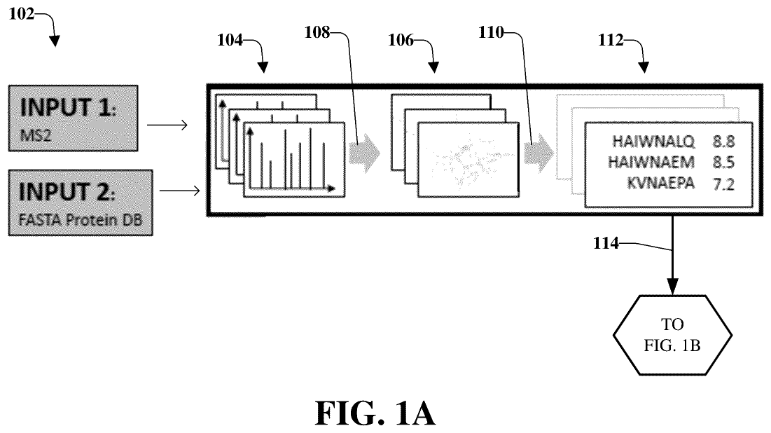

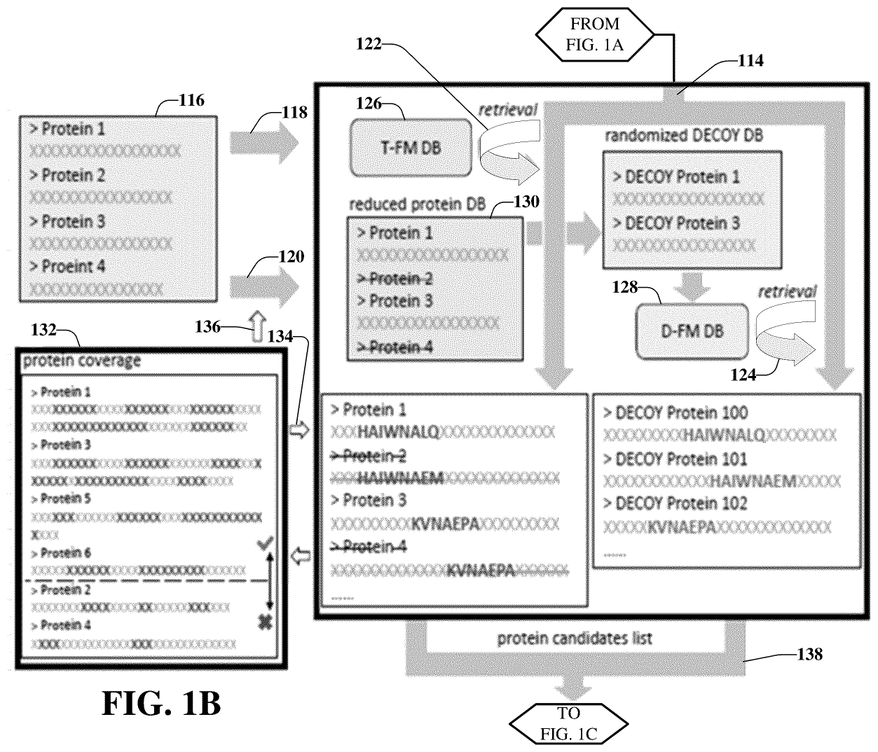

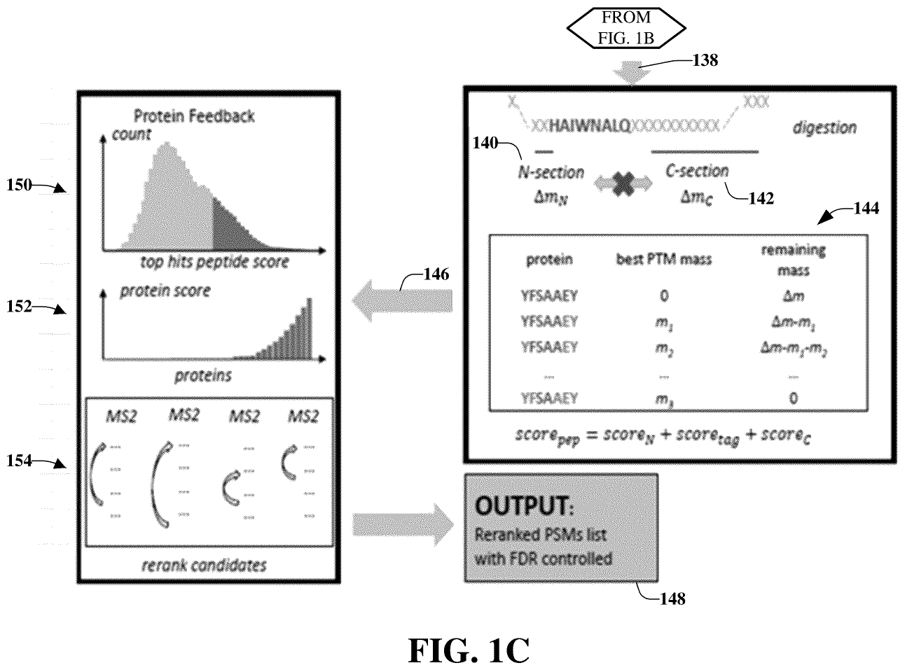

As provided herein, a raw mass spectrometry file is pre-processed and tags are extracted from an MS2 spectra. Then, tags are used to retrieve protein candidates from the FM-indexed database. The mass difference Δm between a theoretical mass of a peptide sequence and the precursor mass of the MS2 spectrum is treated as a total mass shift of potential PTMs, which are then characterized using a greedy approach. Subsequently, peptide candidates with characterized PTM patterns are collected and re-ranked with the protein feedback module. Finally, a PSM list is output with false discovery rate (FDR) controlled by the quality control process. The full workflow of PIPI2 is illustrated in FIGS. 1A though 1C.

FIG. 1A illustrates an example, non-limiting, first stage of a system workflow in accordance with one or more embodiments described herein. Input to the system workflow can include input data 102. For example, the input data 102 can include one or more tandem mass spectra datasets (MS2 spectra) and corresponding databases. The input data 102 of FIG. 1A includes a first input (input 1) and a second input (input 2). In this example, the first input includes MS2 and the second input includes a protein database. Although only two inputs are shown and described, more than two inputs can be received in various implementations.

The input data 102 are converted into directed weighted graphs, illustrated as first graphs 104 and second graphs 106. The first graphs 104 and the second graphs 106 can be different types of graphs. A result of the first graphs 104 can be output, as indicated by main flow arrow 108, and used as inputs to the second graphs 106. A result of the second graphs 106 can be output, as indicated by main flow arrow 110, and can be utilized as inputs in order to be represented as extracted tags 112. The extracted tags 112 are output, as indicated by main flow arrow 114 to FIG. 1B.

FIG. 1B illustrates an example, non-limiting, second stage of the system workflow in accordance with one or more embodiments described herein. Upon or after the extracted tags 112 are generated (and received as inputs from FIG. 1A, as indicated by the main flow arrow 114), tags of various lengths 116 are received, as indicated by main flow arrows 118 and 120. The tags of various lengths 116 are used to retrieve (indicated by arrows 122 and 124) protein candidates from an FM-indexed target protein database (T-FM DB 126) and an FM-indexed decoy protein database (D-FM DB 128). The D-FM DB 128 is generated from a reduced target protein database 130 based on protein coverage 132, received as indicated by side arrows 134 and 136. The resulting protein candidates list is output, as indicated by main flow arrow 138 to FIG. 1C.

FIG. 1C illustrates an example, non-limiting, third stage of the system workflow in accordance with one or more embodiments described herein. As illustrated in FIG. 1C, every protein candidate in the protein candidates list is digested and separated by the tag into N-section 140 and C-section 142. Upon or after the separation, Δm_{N} and Δm_{C} are settled independently in the two sections by a greedy approach, as illustrated at 144. The results are output, as indicated by main flow arrow 146, to determine protein feedback. Finally, candidate lists are reranked using the PFM and the system workflow (e.g., PIPI2) outputs, as indicated by output data 148, a reranked PSM list with FDR controlled.

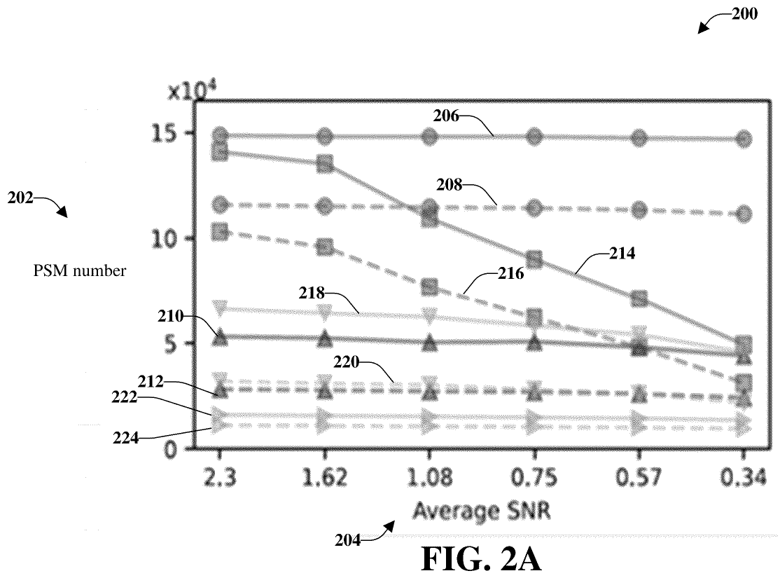

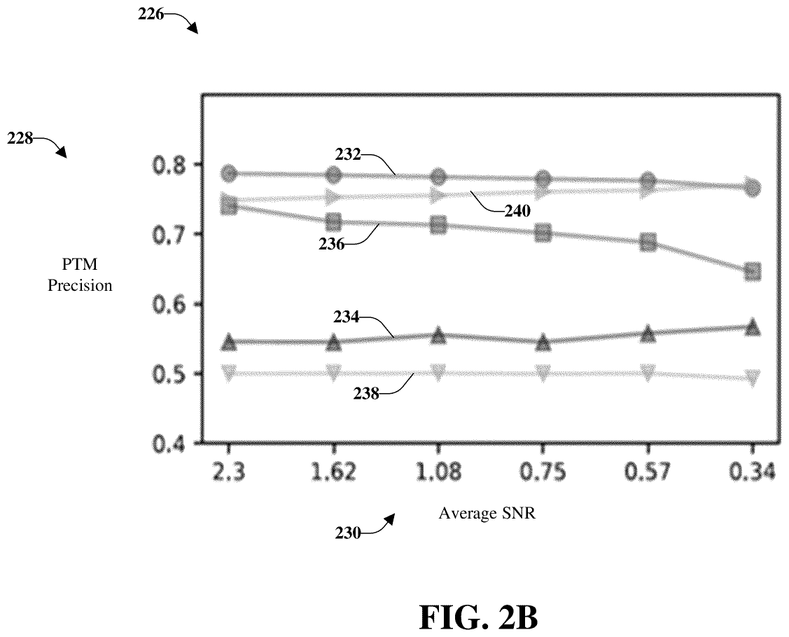

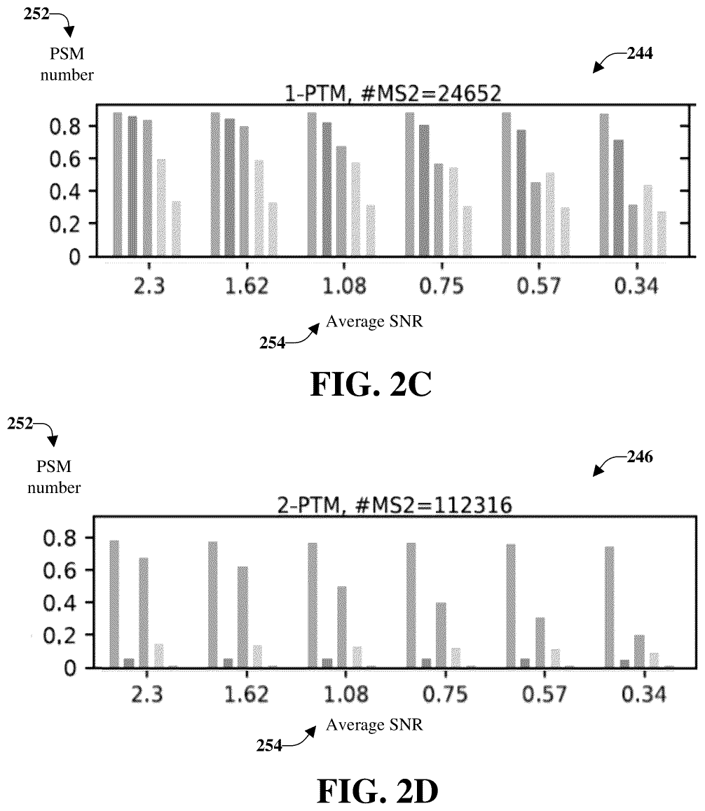

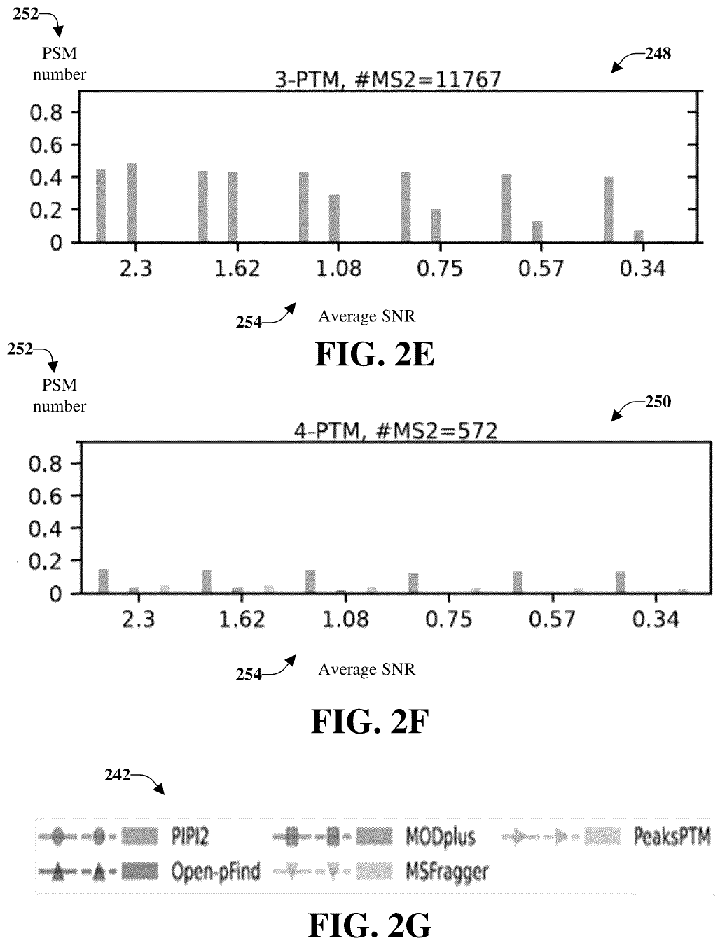

FIG. 2A illustrates a first chart 200 showing performance results of various simulations performed in accordance with one or more embodiments described herein. The various simulations include simulations performed with the system workflow (the disclosed embodiments) and with various conventional systems in order to perform comparison analysis. To search simulated data sets (each set contains 150660 MS2 spectra with up to four PTMs from 18 different PTMs), the system workflow of the disclosed embodiments (e.g., PIPI2) was used. In addition, for comparison purposes, four conventional methods, Open-pFind, MODplus, MSFragger, and PeaksPTM were used.

More specifically, FIG. 2A illustrates results of the numbers of PSM with correct backbone (solid lines) and the number of PSM with correct PTM patterns (dashed lines). In the first chart 200, the PSM number 202 is illustrated on the vertical axis and average signal-to-noise ratio (SNR) 204 is illustrated on the horizontal axis.

In FIG. 2A, line 206 (solid line with circles) indicates results of the numbers of PSM with correct backbone using the disclosed embodiments (e.g., PIPI2). Line 208 (dashed line with circles) indicates results of the number of PSM with correct PTM patterns using the disclosed embodiments.

Line 210 (solid line with triangles) indicates results of the numbers of PSM with correct backbone using Open-pFind. Line 212 (dashed line with triangles) indicates results of the number of PSM with correct PTM patterns using Open-pFind.

Further, line 214 (solid line with squares) indicates results of the numbers of PSM with correct backbone using MODplus. Line 216 (dashed line with squares) indicates results of the number of PSM with correct PTM patterns using MODplus.

Additionally, line 218 (solid line with inverted triangles) indicates results of the numbers of PSM with correct backbone using MSFragger. Line 220 (dashed line with inverted triangles) indicates results of the number of PSM with correct PTM patterns using MSFragger.

Line 222 (solid line with arrows) indicates results of the numbers of PSM with correct backbone using PeaksP™. Line 224 (dashed line with arrows) indicates results of the number of PSM with correct PTM patterns using PeaksP™.

FIG. 2B illustrates a second chart 226 showing performance results of precision of PTM characterization of the various simulations performed in accordance with one or more embodiments described herein.

In the second chart 226, PTM precision 228 is on the vertical axis and average SNR 230 is on the horizontal axis. Illustrated in FIG. 2B is precision of PTM characterization, calculated by the number of PSMs with correct PTM patterns divided by the number of PSMs identified as carrying PTMs.

The precision of PTM characterization for the disclosed embodiments is indicated at line 232 (solid line with circles). The precision of PTM characterization for Open-pFind is indicated at line 234 (solid line with triangles). The precision of PTM characterization for MODplus is indicated at line 236 (solid line with squares). The precision of PTM characterization for MSFragger is indicated at line 238 (solid line with inverted triangles). Lastly, the precision of PTM characterization for PeaksP™ is indicated at line 240 (solid line with arrows).

FIGS. 2C-2F illustrate subplots showing performance results of the sensitivity of PTM characterization of the various simulations performed in accordance with one or more embodiments described herein. FIG. 2G illustrates a legend 242 that provides a guide to the colors, symbols, and lines used for FIGS. 2A through 2F in accordance with one or more embodiments described herein.

Specifically, FIG. 2C illustrates a first subplot 244, FIG. 2D illustrates a second subplot 246, FIG. 2E illustrates a third subplot 248, and FIG. 2F illustrates a fourth subplot 250. In the subplots, PTM sensitivity 252 is illustrated on the vertical axis and average SNR 254 is illustrated on the horizontal axis.

The sensitivity of PTM characterization is calculated by the number of PSMs with correct PTM patterns divided by the ground truth number of MS2 spectra with PTMs (noted in the subplot titles). The results are categorized by the ground truth number of PTMs.

In further detail, for the first subplot 244 (FIG. 2C), there is one PTM, and the number of MS2 spectra is 24652. For the second subplot 246 (FIG. 2D), there are two PTM and the number of MS2 spectra is 112316. For the third subplot 248 (FIG. 2E), there are three PTM and the number of MS2 spectra is 11767. Further, for the fourth subplot 250 (FIG. 2F), there are four PTM and the number of MS2 spectra is 572.

RETRIEVING CANDIDATES USING TAGS OF VARIOUS LENGTHS: With reference again to FIG. 1B, the target FM-indexed protein database (T-FM DB 126) and the decoy FM-indexed protein database (D-FM DB 128) are constructed based on shuffled target protein sequences. Given a tag of any length, protein sequences and relative positions of the tag are retrieved from both the T-FM DB 126 and the D-FM DB 128.

PEPTIDE SCORING AND PTM CHARACTERIZATION: Given a tag and a protein candidate sequence, peptide candidates are generated by digesting the proteins on the N-terminal and C-terminal sides of the tag. The mass difference between the theoretical mass of the peptide sequence and the precursor mass of the spectrum is considered the total mass shift of the PTMs in the peptide. If the tag starts from the N-terminal or ends at the C-terminal of the peptide, it divides the peptide sequence into two sections: the tag section and the rest of the peptide sequence, which is referred to as the N-section or the C-section. Alternatively, when the tag lies in the middle, the peptide sequence is divided into the tag section and both the N-section and C-section, as illustrated in FIG. 1C.

Next, the PTM characterization problem is divided into two independent sub-problems in the N-section and the C-section, with mass shift Δm_{N} or Δm_{C}, as illustrated in FIG. 1C. In each section, a greedy approach is used to characterize PTMs by allocating the mass shift to amino acids. This approach starts from the amino acids at the section's end and moves towards the inner amino acids. Initially, the whole section is set as the potential zone where the amino acids are considered possible to carry PTMs. Then, the potential zone size will be decreased by iteratively removing some amino acids. In each iteration, all the PTMs on amino acids in the potential zone are assessed one by one to find the current best PTM, with which the largest number of experimental peaks are matched to the theoretical peaks. Based on the inter-dependency of the masses of the b and y ions, the potential zone is updated by removing the amino acids which can affect the match to experimental peaks. For example, if Oxidation on S is the best PTM for YFSAAEY that matches the largest number of experimental peaks to theoretical b1, b2, b3, and b4 ions, then YFSA will be removed from the potential zone because any other PTM on them would modify the masses of those four b ions and, therefore, break the match. The iteration terminates when the potential zone is empty or Δm is fully settled. Finally, the peptide candidate with a characterized PTM pattern is assigned a score equal to the sum of intensities of the matched peaks of the tag, N-section, and C-section.

PROTEIN FEEDBACK MODULE: The top candidates (e.g., the top 20 candidates, if available, or another defined number of candidates) of every MS2 spectrum are collected, followed by the estimation of the significance of these candidates through the PFM as illustrated in FIG. 1C. Concretely, the top hits of each MS2 spectrum are ranked by their peptide scores, as indicated at 150, and those above the median are used to calculate the protein scores 152. Then, the 20 peptide candidates (or another defined number of candidates) of each MS2 spectrum are reranked based on the scores of proteins that contain the candidates, resulting in reranked candidates 154. For example, a top candidate could be replaced with a candidate that has a slightly lower peptide score but a higher protein score.

PROTEIN DATABASE REDUCTION: When the protein database is over-complete compared to proteins in the data set, most protein entries in the database hinder the identification by increasing the chance of random matches. The reliability of the existence of the proteins is assessed and the unreliable ones are removed before the search starts. Using extracted tags, target protein candidates are first retrieved from the T-FM DB 126. Then, the coverage of each target protein candidate is evaluated. Tags serve as supporting evidence for the existence of proteins. The more tags supporting a protein, the more reliable the protein is. The protein coverage Cₚ is defined in Equation 1 as follows:

$${C}_{P} = \frac{1}{{L}_{P}} {\sum }_{a = 0}^{{L}_{P}} {S}_{a} . \qquad Equation 1$$

where P denotes a protein; α denotes the indices of amino acids in the protein sequence; Lₚ is the length of protein P measured by the number of amino acids; and S_{α} is the significance of amino acid α.

If all amino acids are 100% significant, Cₚ will be 1. S_{α} is defined by Equation 2 as follows:

$${S}_{a} = 1 - {\prod }_{t ∈ T ( P , a )} ( 1 - \frac{{I}_{a , t}}{\vert P ( t ) \vert} ) \qquad Equation 2$$

$${I}_{a , t} = \frac{{I}_{1 , a , t} + {I}_{2 , a , t}}{2} .$$

where t denotes tags; T (P, α) is the set of tags extracted from all MS2 spectra that retrieve protein P and cover amino acid α, e.g., the tags that cover protein P at the position of amino acid α; |P (t)| is the size of all proteins from the database that contain tag t; and I_{α,t} is the intensity of amino acid α in tag t, calculated as the average of intensities of the two peaks that match amino acid α. The reason that I_{α,t} is normalized by |P(t)| is to emphasize the uniqueness of the tag. The more ambiguous a tag is, the more weakly that ambiguous tag serves as evidence. Target proteins with Cₚ smaller than 10% are treated as unreliable and removed from the candidate lists of all MS2 spectra. The D-FM DB 128 is then built from the remaining proteins. The numbers of target proteins and decoy proteins remain close.

As previously mentioned, peptide identification is important in bottom-up proteomics. PTMs are crucial in regulating cellular activities. Many database search methods have been developed to identify peptides with PTMs and characterize the PTM patterns. However, it is still challenging to identify peptides with PTMs, especially peptides with multiple PTMs. To address this issue, provided herein is a sensitive open search tool, referred to as PIPI2, with much better performance on peptides with multiple PTMs than other methods. With a greedy approach, the PTM characterization problem is simplified from a combinatorial problem into a linear one, which enables characterizing multiple PTMs on one peptide. On the simulation data sets with up to four PTMs per peptide, PIPI2 (using the disclosed embodiments) identified over 90% of the spectra, at least 56% more than five other conventional methods. PIPI2 also characterized these PTM patterns with the highest precision of 77%, demonstrating a significant advantage in handling peptides with multiple PTMs. In the real applications, PIPI2 identified 30% to 88% more peptides with PTMs than conventional methods.

Peptide identification is a fundamental step in bottom-up protcomics. Conventional database search methods require a narrow precursor mass tolerance, such as 10 ppm (part per million), to retrieve peptide sequences from a database as peptide candidates, then calculate the correlation between experimental peaks in tandem mass (MS2) spectra and theoretical peaks generated from the candidates in the database. Such methods are often called closed search methods. However, when an MS2 spectrum contains PTMs, the true peptide sequence might not be retrieved because the mass change brought by the PTMs might shift the precursor mass of the MS2 spectrum out of the tolerance range of the theoretical sequence.

One conventional method conducted an open search with a much larger precursor mass tolerance of ±500 Daltons and identified around 46% more peptides in addition to that identified by closed search. Although open search bypasses the precursor mass shift issue by significantly loosening the precursor mass tolerance, characterizing PTM patterns (e.g., the numbers, types, and sites of PTMs) is still a problem. For example, when using a software search engine that uses mass spectrometry data to identify proteins from peptide sequence databases (e.g., the commercial search engine MASCOT) for database search, users are allowed to pre-specify up to 9 variable PTMs, obviously insufficient compared to the number of PTM entries recorded in one or more comprehensive databases of protein modifications for mass spectrometry applications (e.g., UNIMOD). PTMs are important in regulating cellular activities. It is therefore desirable to identify peptides with PTMs, especially without any prior specification. Therefore, unique challenges exist for identifying the backbones (plain peptide sequence, regardless of PTMs), as well as characterizing the PTM patterns.

Using tags (peptide sequence segments of consecutive amino acids) is a widely-used option for this purpose. Tags are invariant to PTMs because the tags can be extracted from unshifted peaks even when PTMs exist, and retrieves peptide candidates ignoring the precursor mass shift. Since the 2000s, several methods started to utilize tags to characterize particular PTMs of interest, such as phosphorylation. More tag-based methods extended the target to characterize multiple PTMs in peptides without user specification in the 2010's. They first generate a peptide candidate list using tags of a certain fixed length k (the number of amino acids in the tag), noted as tag-k, and then score the candidates and characterize potential PTMs with different strategies.

A conventional technique (e.g., MODplus) uses special signs to concatenate all protein sequences from the protein database into a single string and pre-calculates a hash map from all theoretical tag-3s to their occurrences in the concatenated string. The conventional technique then uses the top 100 tag-3s ranked by the sum of intensities of involved peaks to retrieve all protein candidates from the hash map. Peptide candidates are in silico digested from protein candidates and undergo PTM characterization using dynamic programming in subsequent steps. Another conventional technique (e.g., Open-pFind) also pre-calculates a hash map from all theoretical tag-5s to their occurrences in the protein database. Then, protein candidates retrieved by the top 100 tag-5s are used to generate peptide candidates. All potential single PTM or 2-PTM combinations are exhaustively examined to find the best PTM pattern.

Another conventional approach, referred to as PIPI, encodes MS2 spectra and peptide candidates into sparse vectors with extracted and theoretical tag-3s. The inner products between vectors are used to retrieve the top 20 peptide candidates for PTM characterization. The above noted tag-based methods all rely on a peptide candidate list as the following search space. Obviously, the quality of the peptide candidate list is critical to the performances on both backbone identification and PTM characterization of tag-based methods. Containing PTMs or not, if the true peptide sequence is not included in the list or ranks extremely low, the peptide-spectrum match (PSM) will be wrong.

However, the choice of a suitable tag length is problematic: shorter tags are more sensitive but less accurate because these shorter tags appear in much more peptides; longer tags are more accurate but more likely to be influenced by wrong amino acids extracted from noise peaks. The complexity level of different data sets varies in terms of peptide lengths and the numbers of PTMs in peptides. For MS2 spectra of longer peptides with no PTM, longer tags can be extracted from more unshifted peaks. While for MS2 spectra of shorter peptides with multiple PTMs, only shorter tags are available. Thus, for different data sets, using tags of a fixed length cannot guarantee the quality of the peptide candidate list, resulting in a potential performance drop in backbone identification and PTM characterization.

The embodiments provided herein combine tags of various lengths (tag-V) to improve tag-based methods. Using tag-V, the quality of the peptide candidate list is accommodated for data sets at different complexity levels. The disclosed embodiments can be generally applied in any tag-based method that uses tags to retrieve peptide candidates. Experiments were conducted that used tag-V with the search engine provided herein, referred to as PIPI2, to demonstrate the advantages in performances on backbone identification and PTM characterization, compared to the disclosed embodiments (PIPI2) using different tag-ks, MODplus, and Open-pFind. Using tag-V, the disclosed embodiments (PIPI2) output 35% more backbone identifications under the same quality control and 49% more PTM characterizations with 7% higher precision, than the other methods on all data sets with or without PTMs.

Given an MS2 spectrum (e.g., input data 102), an entire m/z range was divided into subranges of 100 Da, and the intensities of peaks within each subrange were normalized by dividing the intensities of peaks by the highest intensity in that subrange. A weighted directed graph G was then constructed for tag extraction. The m/z difference between any two peaks was calculated to match potential amino acids. When an amino acid was matched, two nodes and a directed edge were added to G, for example, nᵢ weighted by Iᵢ (intensity of peak i), nⱼ weighted by Iⱼ (intensity of peak j), e_{i,j} weighted by Iᵢ+Iⱼ. All paths from nodes of zero in-degree to nodes of zero out-degree as long as possible, were extracted as tags using a depth-first search. A depth-first search is a process for searching (or traversing) data structures (e.g., tree data structures, tree or graph data structures), starting at the root node.

When fixing tag length k, tag-ks were kept, tags shorter than k were discarded, and tags longer than k were divided into several tag-ks. When combining tags of various lengths, tags of lengths ranging from 3 to 9 (e.g., between 3 amino acids and 9 amino acids) were kept, tags shorter than 3 (e.g., 3 amino acids) were discarded, and tags longer than 9 (e.g., 9 amino acids) were divided into several tag-9s. Finally, tags were ranked by the sum of the intensities of peaks involved.

Candidates are retrieved from an indexed database of peptide sequences or protein sequences. For example, the indexed database is an FM-indexed database, which is a compressed full-text substring index. The FM-index is a data structure based on Burrows-Wheeler Transform with some auxiliary data structures. Given a tag of any length, all occurrences of the tag can be located in the database in O(N) time, where N is the number of the occurrences. Unlike the pre-calculated hash maps of Open-pFind and MODplus with keys of tags of single fixed length, FM-index allows for queries of tags of different lengths. This flexibility enables the usage of tag-V.

Using either tag-V or any fixed tag-k, the top 100 tags were used to build the peptide candidate list. The peptide candidates were ranked by score S calculated as:

$$S = {\sum }_{t ∈ T} \frac{{S}_{t}}{{N}_{t}} . \qquad Equation 3$$

where T is the set of the top 100 tags, S, is the score of tags, and N, is the number of occurrences of tag t in the database as a normalization.

To demonstrate the benefit of tag-V in a real pipeline, tag-V and tag-ks (k ranges from 3 to 9) were implemented with the disclosed embodiments (PIPI2) and the result was compared with MODplus and Open-pFind results, since both MODplus and Open-pFind also used fixed tag lengths.

Experiments and Results: Two experiments were conducted to demonstrate the benefit of using tag-V, on data sets with or without PTMs. In experiment 1, the quality of the peptide candidate lists built by tag-V and seven different tag-ks were compared. In experiment 2, the performance of the disclosed embodiments, referred to as PIPI2 (labeled as PIPI2-V) on backbone identification and PTM characterization was evaluated and compared it with PIPI2 using seven different tag-ks (labeled as PIPI2-k), MODplus (2.01), and Open-pFind (3.2.0).

Date Sets: A first group of data sets, identified as H01 to H06, [was from ProteomeTools (on Proteome Xchange Consortium with data identifier PXD004732)] consisting of 123 pools of synthesized tryptic human peptides of different average lengths with small standard deviations. From pools 1, 21, 41, 61, 81, and 101, and the corresponding identification results provided by the authors using MaxQuant, data sets H01 to H06 were generated with ground truth. In each data set, MS2 spectra with scores larger than 50 were selected and the identified peptide sequences were recorded as ground truth. These true peptides combined with peptides from E. coli proteins peptides as entrapment were used as the peptide database. Further, data set Hmix were generated by mixing peptides of different lengths from all 123 pools as a more realistic case than using peptides of single length only.

The second data set, SIMU, was simulated by AlphaPeptDeep with parameters as indicated in Table I below.

TABLE I
PARAMETERS SET FOR MS2 SPECTRA
PREDICTION BY ALPHAPERTDEEP

| | |
|---|---|
| Enzyme | trypsin |
| Instrument | lumos |
| Normalized collision energy | 30 |
| Max variable modifications | 4 |
| Max missed cleavages | 2 |
| Min precursor charge | 2 |
| Max precursor charge | 4 |
| Min peptide length | 6 |
| Max peptide length | 35 |
| Fix modification | Carbamidomethyl on C |

Variable modifications

| | |
|---|---|
| Fluoro on A | Malonyl on S |
| Sulfide on D | Methylamine on T |
| Decarboxylation on E | Carbonyl on V |
| Nitro on F | Chlorination on W |
| Deamidated on N | Phospho on Y |
| Dioxidation on P | Oxidation on G |
| Deoxyhypusine on Q | Carboxyethyl on H |
| Ethanolyl on R | Carbonyl on L |
| Oxidation on M | |

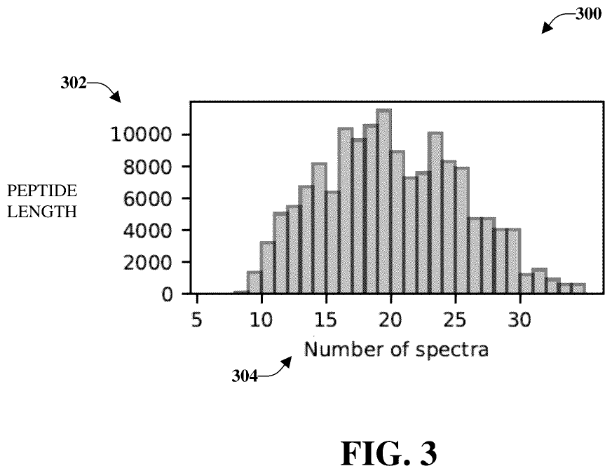

Using protein “Packet Kmod” in PXD009449 as a template, data set SIMU was generated consisting of 150,660 MS2 spectra with 0 to 4 PTMs selected from 17 common PTMs. The protein database contains the template protein and E. coli proteins as entrapment. The peptide length distribution of the data set SIMU is illustrated in FIG. 3, where peptide length 302 is illustrated on the vertical axis and number of spectra 304 is illustrated on the horizontal axis.

A third group of data sets, F01 to F12 (was from ProteomeTools, on ProteomeXchange Consortium with data identifier PXD010595), of synthetic human peptides isoforms of different average lengths with small standard deviation. Isoform pools 1, 11, 21, . . . , 111 were taken, as data sets F01 to F12. The protein database contained the synthetic protein template provided with the data and E. coli protein database as entrapment. The peptide length information and numbers of MS2 spectra in data sets F01 to F12 are shown in Table II below.

TABLE II
PEPTIDE LENGTHS AND NUMBERS
OF MS2 SPECTRA IN F01 TO F12

| Data ID | Average | Standard deviation | # MS2 spectra |
|---|---|---|---|
| F01 | 7.44 | 1.07 | 52,672 |
| F02 | 7.65 | 1.22 | 51,045 |
| F03 | 8.20 | 0.80 | 53,473 |
| F04 | 9.14 | 0.54 | 53,926 |
| F05 | 10.06 | 0.28 | 57,541 |
| F06 | 10.98 | 0.16 | 56,877 |
| F07 | 11.92 | 0.27 | 59,164 |
| F08 | 12.83 | 0.54 | 58,375 |
| F09 | 13.77 | 0.79 | 59,010 |
| F10 | 15.61 | 1.31 | 55,795 |
| F11 | 16.55 | 1.57 | 55,106 |
| F12 | 18.43 | 2.12 | 49,671 |

Performance Metrics: For experiment 1 using the data sets H01 to H06, Hmix, and SIMU, the quality of the peptide candidate list was measured by the retrieve rate R, which is defined as the proportion of MS2 spectra whose true peptides were included in the peptide candidate list, and the rank r of the true peptides in the list if retrieved. Different tag-based methods may set different thresholds of rank r to control the number of candidates. The higher the true peptide ranks results in better performance.

For experiment 2 using the data sets F01 to F12, the target-decoy strategy was used to estimate the false discovery rate (FDR). Among PSMs with FDR<0.01, the sensitivity of backbone identification was represented by the identification rate, e.g., the number of MS2 spectra matched to synthesized peptides divided by the number of total MS2 spectra in the data set.

As for experiment 2, using the data set SIMU with known ground truth of every MS2 spectrum, the false discovery proportion (FDP) and output PSMs with FDP<0.01 were directly calculated for all the conventional methods simulated instead of using the target-decoy strategy. However, decoy proteins were still added as extra entrapment proteins for PIPI2, MODplus, and Open-pFind because a decoy database was mandatory for Open-pFind. First, the sensitivity of backbone identification represented by the number of PSMs with the correct backbone N_{b} were measured. Among these PSMs, the sensitivity of PTM characterization by the number of PSMs with correct PTM patterns Nₚ were evaluated. The precision of PTM characterization, Pₚ, was calculated as Nₚ/Nₜₒₜₐₗ, where Nₜₒₜₐₗ is the total number of PSMs identified as containing PTMs. MS2 spectra with 0 PTM were also considered in the PTM characterization performance calculation because a PTM pattern assigned to such MS2 spectra should decrease the performance.

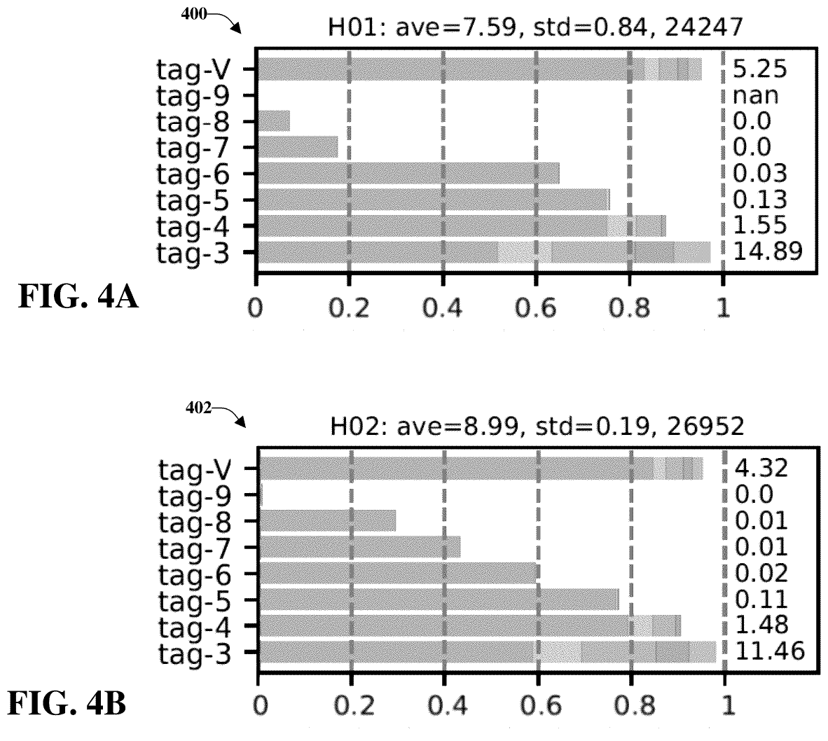

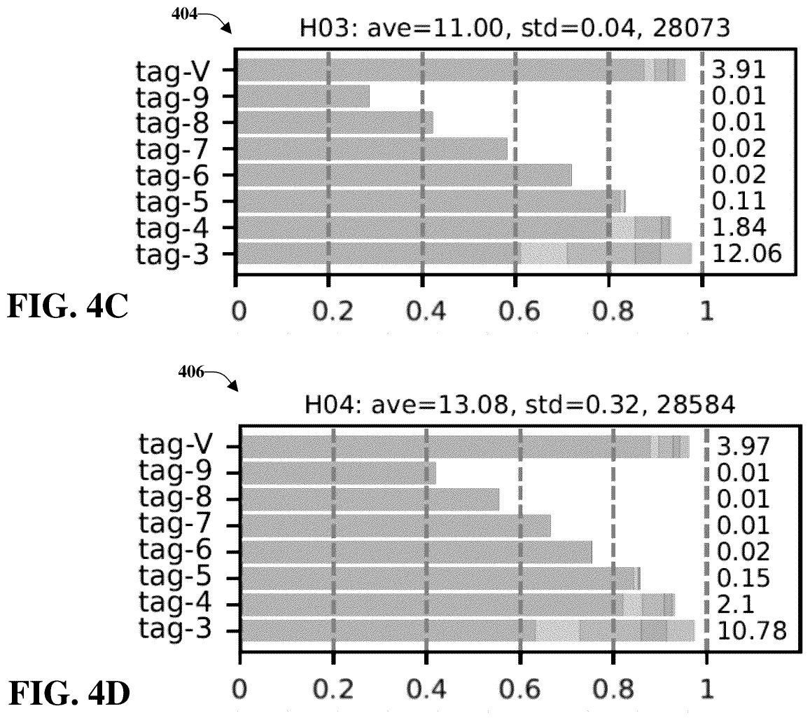

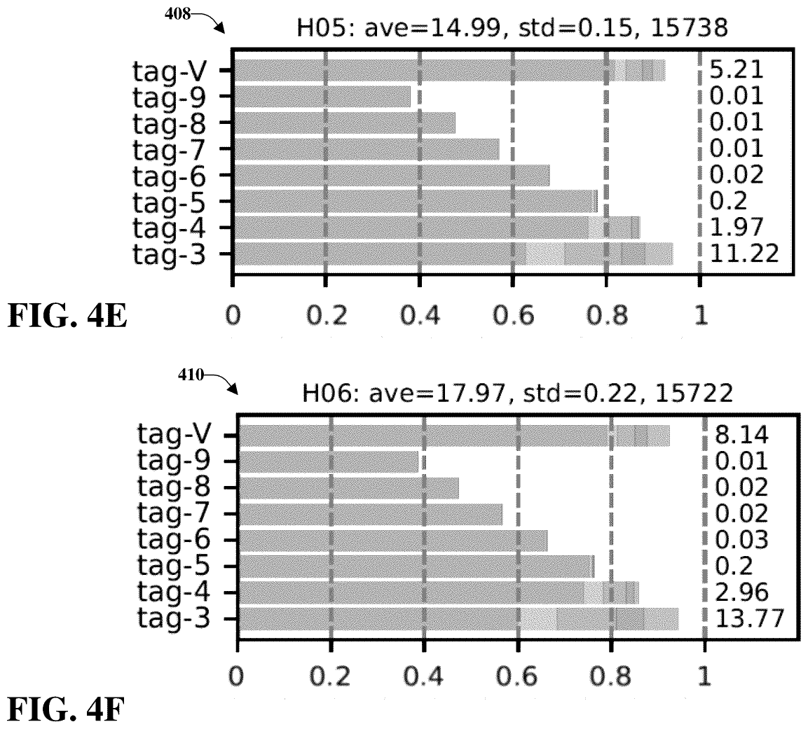

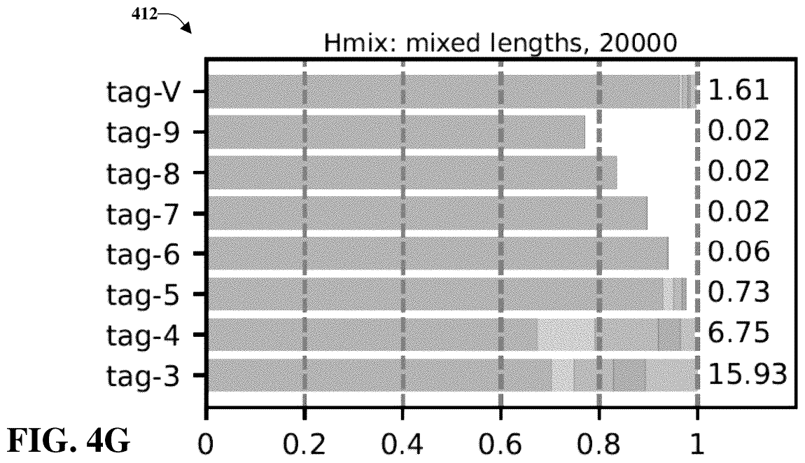

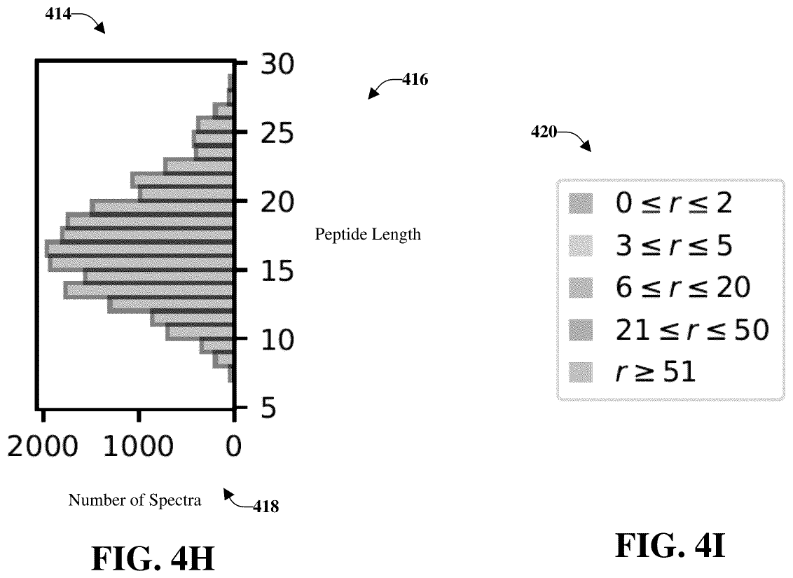

Results of Experiment 1: FIGS. 4A-4G illustrate subplots of the quality of peptide candidate list retrieved by tag-V or tag-ks for data sets H01 to H06, and Hmix, respectively, according to one or more simulations performed in accordance with one or more embodiments described herein. In the subplots (FIGS. 4A-4G), the horizontal axis indicates the proportion of spectra with different ranks (r) of the true peptide. Further, for respective tags tag in the PTM set, the numbers to the right of the bars are the average rank of true peptides whose r<400.

Specifically, FIG. 4A illustrates a first subplot 400 of the peptide candidate list quality of data sets H01. A second subplot 402 of the peptide candidate list quality of data sets H02 is illustrated in FIG. 4B. Further, FIG. 4C illustrates a third subplot 404 of the peptide candidate list quality of data sets H03. A fourth subplot 406 of the peptide candidate list quality of data sets H04 is illustrated in FIG. 4D. A fifth subplot 408 of FIG. 4E illustrates the peptide candidate list quality of data sets H05. FIG. 4F illustrates a sixth subplot 410 of the peptide candidate list quality of data sets H06. In addition, FIG. 4G illustrates a seventh subplot 412 of the peptide candidate list quality of Hmix, which is the proportion of spectra with different ranks (r) of the true peptide.

Subplot titles are the data set identifier (ID), average deviation, and standard deviation of the peptide length, and the number of MS2 spectra in each data set. In further detail, for the first subplot 400 (FIG. 4A), the data set ID is H01, the average deviation is 7.59, the standard deviation is 0.84, and the number of MS2 spectra is 24247. For the second subplot 402 (FIG. 4B), the data set ID is H02, the average deviation is 8.99, the standard deviation is 0.19, and the number of MS2 spectra is 26952. For the third subplot 404 (FIG. 4C), the data set ID is H03, the average deviation is 11.00, the standard deviation is 0.04, and the number of MS2 spectra is 28073. For the fourth subplot 406 (FIG. 4D), the data set ID is H04, the average deviation is 13.08, the standard deviation is 0.32, and the number of MS2 spectra is 28584. For the fifth subplot 408 (FIG. 4E), the data set ID is H05, the average deviation is 14.99, the standard deviation is 0.15, and the number of MS2 spectra is 15738. For the sixth subplot 410 (FIG. 4F), the data set ID is H06, the average deviation is 17.97, the standard deviation is 0.22, and the number of MS2 spectra is 15722. Further, for the seventh subplot 412 (FIG. 4G), the data set ID is Hmix, the tags have mixed lengths, and the MS spectra is 20000.

FIG. 4H illustrates a histogram 414 of the peptide length distribution for Hmix according to one or more simulations performed in accordance with one or more embodiments described herein. The peptide length 416 is illustrated on the vertical axis and the number of spectra 418 is illustrated on the horizontal axis. FIG. 4I illustrates a legend 420 that provides a guide to the colors, symbols, and/or lines used for FIGS. 4A through 4G in accordance with one or more embodiments described herein. The numbers to the right of the bars are the average rank of true peptides whose r<400, where r is the rank of the true peptide in the peptide candidate list.

On all data sets, shorter tags, such as tag-3, had a high retrieve rate of R>0.95 but the true peptide rank r ranged variously from 0 to more than 50 with an average value >10. This was consistent with the observation that shorter tags were more sensitive but less accurate. With longer tags, such as tag-6 to tag-9, R decreased severely, but r was more concentrated between 0 to 2, with an average value close to 0. It showed that longer tags were more accurate but more likely to be absent because of missing peaks. While the results of tag-V combined the sensitivity of shorter tags and the accuracy of longer tags. Using tag-V, the number of MS2 spectra with 0<r<2 was more than that of using any fixed tag-k, and the retrieve rate R was comparable to the most sensitive tag length, e.g., tag-3.

From data sets H01 to H06, the retrieve rate R of longer tags increased because more tags could be extracted from MS2 spectra of longer peptides. For data sets H01 and H02 (FIGS. 4A and 4B, respectively) whose average peptide lengths were smaller than 9, there was no output using tag-9. As for the data set Hmix (FIG. 4G), the performances of different tag-ks were more balanced because Hmix contained peptides of different lengths preferred by different tag-ks. It is noted that tag-V still outperformed all tag-ks with better peptide candidate lists for data sets with unmodified peptides of different lengths.

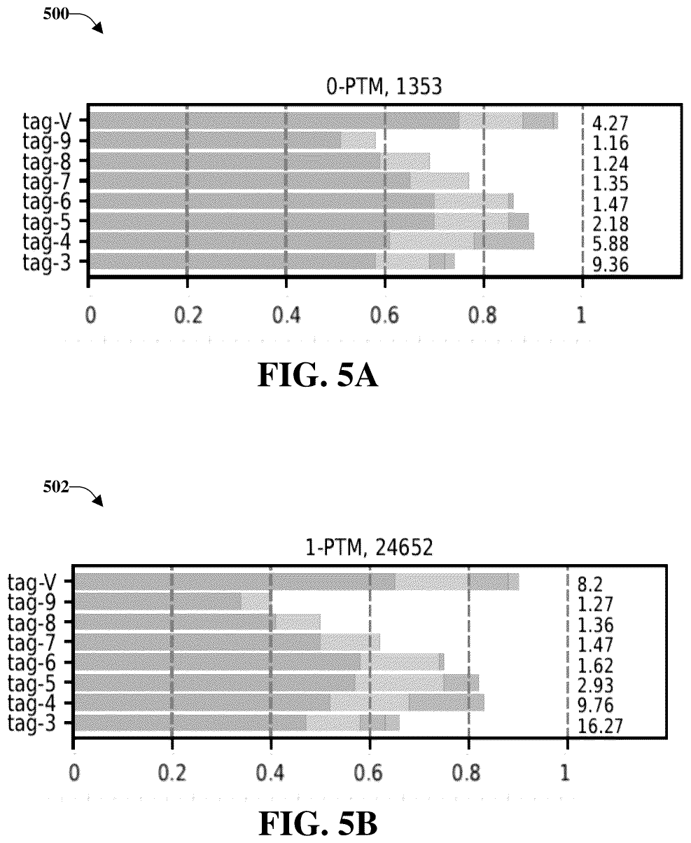

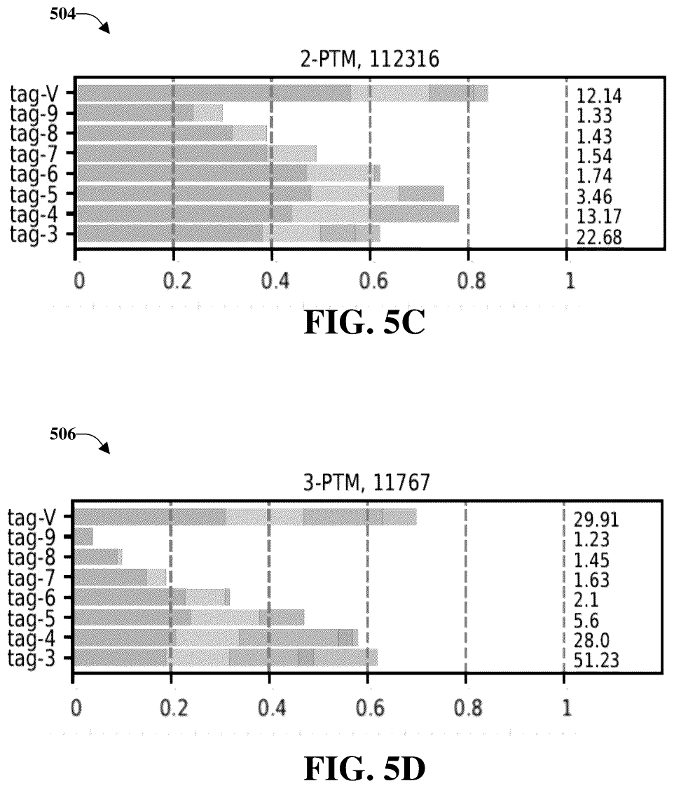

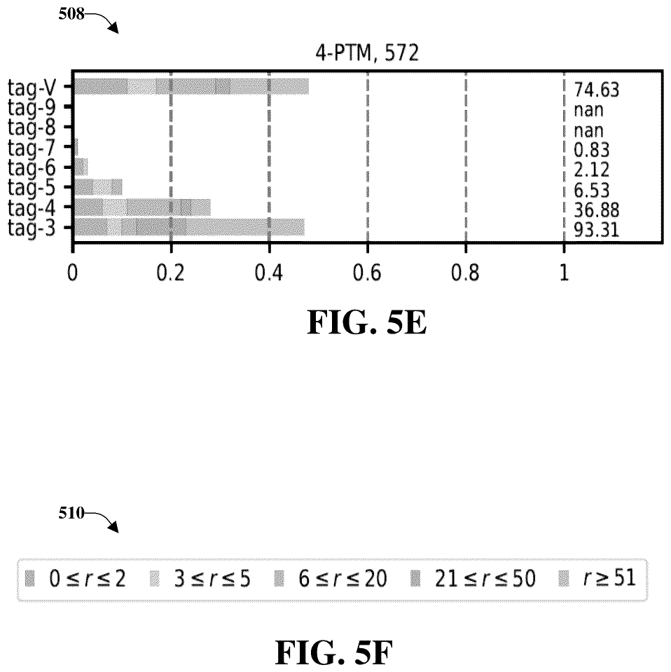

FIGS. 5A to 5E illustrate subplots of the quality of peptide candidate lists retrieved for data set SIMU according to one or more simulations performed in accordance with one or more embodiments described herein. Specifically, FIG. 5A illustrates a first subplot 500 of the quality of peptide candidate list for 0-PTM. FIG. 5B illustrates a second subplot 502 of the quality of peptide candidate list for 1-PTM. A third subplot 504 of the quality of peptide candidate list for 2-PTM is illustrated in FIG. 5C. FIG. 5D illustrates a fourth subplot 506 of the quality of peptide candidate list for 3-PTM. A fifth subplot 408 of the quality of peptide candidate list for 4-PTM is illustrated in FIG. 5E. Further, FIG. 5F illustrates a legend 510 that provides a guide to the colors, symbols, and/or lines used for FIGS. 5A-5E in accordance with one or more embodiments described herein.

Subplot titles are the number of PTMs on the MS2 spectra and the number of such MS2 spectra in the data set. In further detail, for the first subplot 500 (FIG. 5A), the number of PTMs on the MS2 spectra is 0 and the number of such MS2 spectra in the data set is 1353. For the second subplot 502 (FIG. 5B), the number of PTMs on the MS2 spectra is 1 and the number of such MS2 spectra in the data set is 24652. For the third subplot 504 (FIG. 5C), the number of PTMs on the MS2 spectra is 2 and the number of such MS2 spectra in the data set is 112316. For the fourth subplot 506 (FIG. 5B), the number of PTMs on the MS2 spectra is 3 and the number of such MS2 spectra in the data set is 11767. For the fifth subplot 508 (FIG. 5C), the number of PTMs on the MS2 spectra is 4 and the number of such MS2 spectra in the data set is 572.

The results are shown separately in the five subplots of FIGS. 5A-5E, categorized by the true number of PTMs in each MS2 spectrum. For MS2 spectra with different numbers of PTMs, tag-V was always the most sensitive and accurate one among all tag length options shown by the highest retrieve rate and the largest number of MS2 spectra with high-ranking true peptides. With increasing numbers of PTMs in MS2 spectra, it was obvious that the retrieve rate R decreased severely. For MS2 spectra with 4 PTMs (FIG. 5E), there was even no output using tag-8 and tag-9. Besides, a similar trend was observed to that in the data set without PTM. Longer tags lead to a higher average rank of true peptides but a smaller retrieve rate for all MS2 spectra, and shorter tags such as tag-4 had the highest retrieve rate for MS2 spectra with 0 to 2 PTMs and tag-3 for MS2 spectra with 3 to 4 PTMs. Most of the conventional tag-based methods for identifying peptides with PTMs are strongly limited by the number of PTMs in peptides. Some of these conventional methods claim the ability to identify peptides with unlimited numbers of PTMs but the performances for peptides with more than 2 PTMs are far from satisfactory. A peptide candidate list of good quality is a necessary condition for good performance.

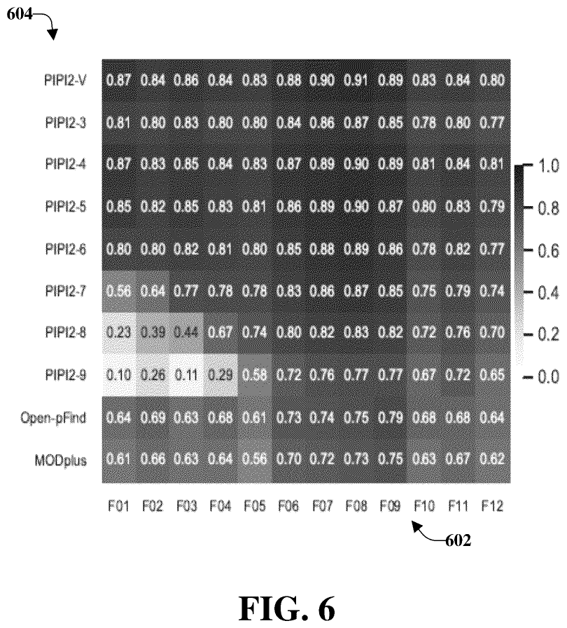

Results of Experiment 2: The results of the data sets F01 to F12 are depicted in FIG. 6, according to one or more simulations performed in accordance with one or more embodiments described herein. Specifically, FIG. 6 illustrates the identification rate of data sets F01 to F12 (indicated on the horizontal axis 602) by PIPI2-V, PIPI2-3 to PIP12-9, MODplus, and Open-pFind (indicated on the vertical axis 604).

Across all 12 data sets (data sets F01 to F12), PIPI2-V had the highest identification rate ranging from 83% to 91% among all the other simulations (e.g., conventional methods), especially 17% and 20% higher than Open-pFind and MODplus on average. Open-pFind and MODplus were only better than PIPI2-7 to PIPI2-9 on data sets F01 to F04 because peptides of shorter lengths were not preferred by longer tags. Apart from that, their performances were worse than all versions of PIPI2-k. On different data sets, the identification rate of each software program increased first and then decreased. The maximum value was generally reached at data sets F08 or F09. PIPI2-9 had correct identifications on F01 even though the average peptide length was shorter than 9. These identifications were from peptides of length 11 for quality control in each data set. With increasing tag length, the performance of PIPI2-ks showed that shorter tags preferred shorter peptides and longer tags preferred longer peptides. These results demonstrated that PIPI2 benefited from combining tags of various lengths and outperformed MODplus and Open-pFind on peptides without PTM.

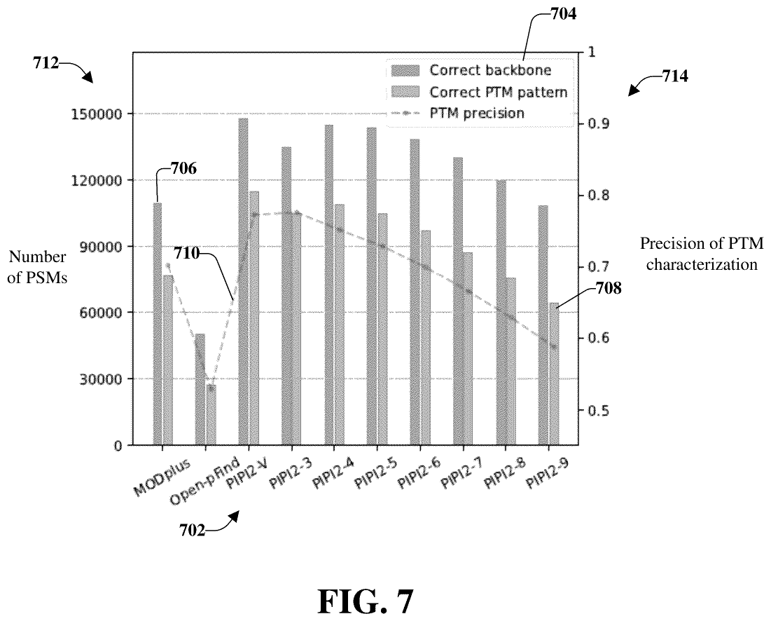

The overall results of the data set SIMU are depicted in FIG. 7 according to one or more simulations performed in accordance with one or more embodiments described herein. The results of data set SIMU are identified by PIPI2-V, PIPI2-3 to PIPI2-9, MODplus, and Open-pFind, as indicated on the horizonal axis 702. As indicated by the legend 704 at the top right side of FIG. 7, the darker colored bars (one of which is identified as 706) represent the numbers of PSMs with correct backbones, N_{b}. The lighter colored bars (one of which is identified as 708), and which are shorter than the darker colored bars for each set, represent the numbers of PSMs with correct backbones and correct PTM patterns, Nₚ. Additionally, the dashed line 710 represents PTM characterization precision of all MS2 spectra, Pₚ.

The number of PSMs 712 are represented on the left vertical axis and the precision of PTM characterization 714 is represented on the right vertical axis.

Among output PSMs with FDP<0.01, N_{b}, Nₚ, and Pₚ were calculated as described above. Compared with MODplus and Open-pFind, PIPI2-V had much more output in terms of N_{b} and Nₚ with higher Pₚ. The percentages by which PIPI2-V exceeded them are shown in Table III below.

TABLE III
PERFORMANCE ADVANTAGES OF PIPI2
OVER MODPLUS AND OPEN-PFIND

| | N_{b} | Nₚ | Pₚ |
|---|---|---|---|
| PIPI2-V over MODplus | 35% | 49% | 7% |
| PIPI2-V over Open-pFind | 193% | 320% | 24% |

The reason that Open-pFind had poor performance was its “tolerance” on user-specified enzyme specificity and max missed cleavages. It output many PSMs that violated user-specified parameters yet with relatively high scores, which might be a strategy for correcting users' inappropriate parameter setting(s). While these two parameters were strictly applied in AlphaPeptDeep, Open-pFind failed to distinguish peptide candidates with different enzyme specificities or numbers of missed cleavages in many PSMs. Such PSMs were removed from Open-pFind's results to make its performance better for a fair comparison. The performance of MODplus was comparable to that of PIPI2-8 and PIPI2-9 while Open-pFind was much lower. Compared with PIPI2-ks, PIPI2-V outperformed all versions in terms of N_{b} and Nₚ, with slightly lower Pₚ than PIPI2-3. In other words, PIPI2-V had the highest sensitivity at the same precision (guaranteed by FDP) level of backbone identification; and the highest sensitivity and precision of PTM characterization. Although in the real pipeline, the performance advantage did not solely come from combining tag-V, it is demonstrated that peptide candidate lists with better quality achieved by tag-V indeed benefited PIPI2 based on the performance comparison between PIPI2-V and all PIPI2-ks.

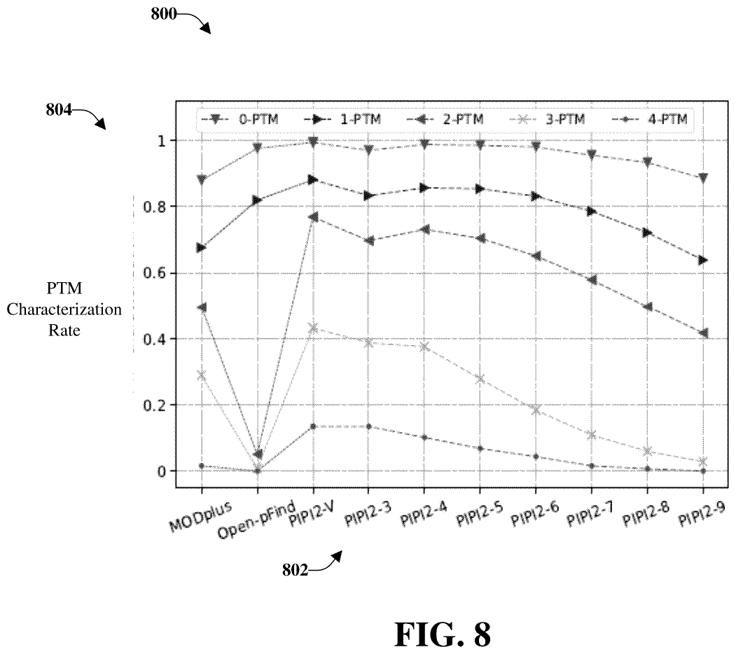

A deeper investigation was conducted into the performance on PTM characterization by categorizing the lighter colored bars (710 of FIG. 7) according to the true number of PTMs in each MS2 spectrum and normalizing them as characterization rate, as depicted in FIG. 8. Specifically, FIG. 8 illustrates a chart 800 of the proportion of PSMs with correct backbones, and PTM patterns from MS2 spectra with 0 to 4 PTMs in data set SIMU, identified by PIPI2-V, PIPI2-3 to PIPI2-9, MODplus, and Open-pFind (indicated on the horizontal axis 802). The PTM characterization rate 804 is illustrated on the vertical axis.

PIPI2-V had the highest PTM characterization rates on all MS2 spectra with 0 to 4 PTMs. For MS2 spectra with 0 or 1 PTM, the characterization rates of all competitors were higher than 0.6. While for MS2 spectra with more than one PTM, Open-pFind could not maintain a good performance, especially for MS2 spectra with 3 or 4 PTMs which accounted for about 83% of all the MS2 spectra in the data set SIMU. This was the reason that Open-pFind had bad performances on SIMU. MODplus performed badly on MS2 spectra with 4 PTMs. Overall, PIPI2-V had the best sensitivity for peptides with different numbers of PTMs and was the only one that could manage peptides with 4 PTMs.

As provided herein, the disclosed embodiments combine tags of various lengths for tag-based methods. The disclosed embodiments serve as a simple and effective solution to the problem of choosing a suitable tag length for retrieving peptide candidates in data sets of different complexity, such as, for example, MS2 spectra with different peptide lengths or different abundance of PTMs. Compared to tags of fixed lengths, using tags of various lengths combines the advantages of the sensitivity of shorter tags and the accuracy of longer tags to obtain a better quality peptide candidate list. These advantages result in better performances on backbone identification and PTM characterization when applied in a real pipeline. This is verified by comparing PIPI2 with tags of various lengths, different single fixed tag lengths, MODplus, and Open-pFind on data sets with or without PTMs.

As discussed herein, peptide identification provides key information for protein inference in bottom-up proteomics. PTMs are essential to understand cellular activities at the protein level. In conventional database search methods for peptide identification, precursor mass is a critical parameter to narrow down the search space. However, true peptides may be excluded from the search space if precursor masses are modified by PTMs. Thus, many researchers use peptide sequence segments, referred to as tags, which are invariant to PTMs in database search. Shorter tags are more sensitive but less accurate, whereas longer tags are more accurate but less frequent. Conventional methods use tags of fixed lengths, ignoring the effect of different tag lengths. To address this issue, the disclosed embodiments combine tags of various lengths to improve tag-based peptide identification methods. Using combined tags, true peptides are included in the search space in more cases, resulting in at least 35% and 49% more peptide identifications and PTM results compared to benchmark methods using the same quality control parameters.

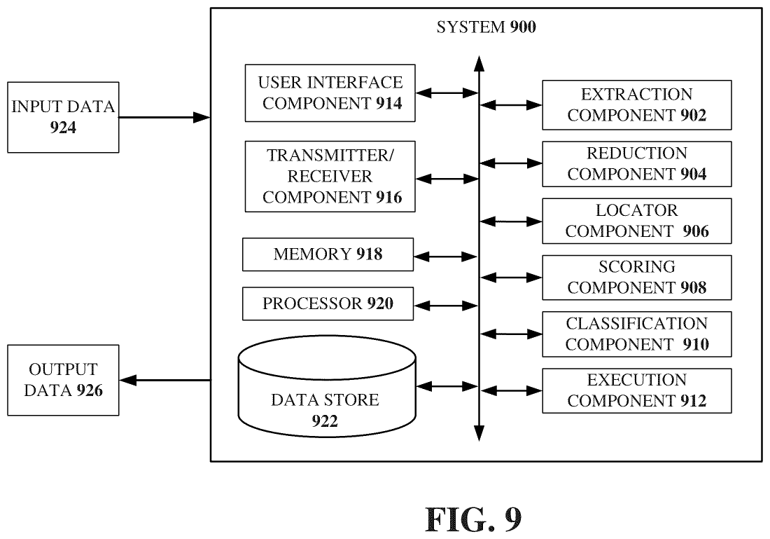

FIG. 9 illustrates an example, non-limiting, system 900 for facilitating using a greedy approach to identify peptides with multiple post-translational modification in accordance with one or more embodiments described herein. Repetitive description of like elements employed in other embodiments described herein is omitted for sake of brevity. The system 1400 can comprise one or more of the components and/or functionality of system workflow of FIGS. 1A-1C and/or the computer-implemented methods discussed herein, and vice versa.

Aspects of systems (e.g., the system 900 and the like), devices, apparatuses, and/or processes explained in this disclosure can constitute machine-executable component(s) embodied within machine(s) (e.g., embodied in one or more computer readable mediums (or media) associated with one or more machines). Such component(s), when executed by the one or more machines (e.g., computer(s), computing device(s), virtual machine(s), and so on) can cause the machine(s) to perform the operations described.

In various embodiments, the system 900 can be any type of component, machine, device, facility, apparatus, and/or instrument that comprises a processor and/or can be capable of effective and/or operative communication with a wired and/or wireless network. Components, machines, apparatuses, devices, facilities, and/or instrumentalities that can comprise the system 1400 can include tablet computing devices, handheld devices, server class computing machines and/or databases, laptop computers, notebook computers, desktop computers, cell phones, smart phones, consumer appliances and/or instrumentation, industrial and/or commercial devices, hand-held devices, digital assistants, multimedia Internet enabled phones, multimedia players, and the like.

As illustrated, the system 900 can include an extraction component 902, a reduction component 904, a locator component 906, a scoring component 908, a classification component 910, an execution component 912, a user interface component 914, a transmitter/receiver component 916, at least one memory 918, at least one processor 920, and at least one data store 922. The at least one memory 918 can store computer executable components and instructions. The at least one processor 920 can facilitate execution of the instructions (e.g., computer executable components and corresponding instructions) by the extraction component 902, the reduction component 904, the locator component 906, the scoring component 908, the classification component 910, the execution component 912, the user interface component 914, the transmitter/receiver component 916, and/or other system components.

As depicted, in some embodiments, one or more of the extraction component 902, the reduction component 904, the locator component 906, the scoring component 908, the classification component 910, the execution component 912, the user interface component 914, the transmitter/receiver component 916, the at least one memory 918, the at least one processor 920, and the at least one data store 922 can be electrically, communicatively, and/or operatively coupled to one another to perform one or more functions of the system 900.

The system 900 receives input data 924 (e.g., the input data 102) via, for example, the transmitter/receiver component 916. In an implementation, the input data 924 can include one or more tandem mass spectra datasets. According to some implementations, the input data 924 can include one or more tandem mass spectra (MS2 spectra) datasets and corresponding databases. In an example, the tandem mass spectra can be obtained from a group of proteins with post-translational modifications.

Based at least in part on the input data 924, the extraction component 902 can extract tags that represent sequential amino acids based on peaks in tandem mass spectra being converted into a weighted directed graph, resulting in extracted tags. According to some implementations, the extracted tags can include tags of different lengths.

Using the extracted tags, the reduction component 904 can reduce a protein database. For example, to reduce the protein database, the reduction component 904 can remove, from the protein database, extracted tags determined to have a reliability level that is below a reliability threshold level, The reliability level can be based on a determination of respective coverages of proteins in the protein database.

The locator component 906 can locate a tag in an indexed database, resulting in a located tag. The extracted tags can include the located tag. Proteins that comprise the located tag can be scored by the scoring component 908. The localization of the tag in the indexed database can be performed by the locator component 906 without user specification.

Based on the score assigned by the scoring component 908, the classification component 910 can characterize or classify post-translational modification patterns. For example, the classification component 910 can use a greedy approach process to characterize the post-translational modification patterns.

Based on a result of the greedy approach process, the execution component 912 can facilitate implementation of a quality control process. In some implementations, output data 926 (e.g., the output data 148) can be provided (e.g., via the transmitter/receiver component 916, via the user interface component 914) and can include a reranked PSM list with FDR being controlled. For example, the target-decoy strategy can be used to estimate the FDR.

According to some implementations, prior to the locator component 906 locating the tag in the indexed database, the index database can be generated (e.g., via a generation component (not shown)). For example, at least one T-FM DB (e.g., the T-FM DB 126) and/or at least one D-FM DB (e.g., the D-FM DB 128) can be generated as discussed herein. The indexed database can facilitate protein candidate retrieval using tags of different lengths.

The system 900 (e.g., via the user interface component 914, via the transmitter/receiver component 916, or via another system component) can perform real-time updates related to the quality control process and/or other output data (e.g., the output data 926) that are transmitted to a client device (e.g., user equipment). The real-time updates can cause the client device to activate a user interface with the time critical updated information so the user becomes aware of the update in real-time.

According to some implementations, the user interface component 914 (as well as other interface components discussed herein) can provide a Graphical User Interface (GUI), a command line interface, a speech interface, Natural Language text interface, and the like. For example, a GUI can be rendered that provides an entity with a region or means to load, import, select, read, and so forth, various requests and can include a region to present the results of the various requests. These regions can include known text and/or graphic regions that include dialogue boxes, static controls, drop-down-menus, list boxes, pop-up menus, edit controls, combo boxes, radio buttons, check boxes, push buttons, graphic boxes, and so on. In addition, utilities to facilitate the information conveyance, such as vertical and/or horizontal scroll bars for navigation and toolbar buttons to determine whether a region will be viewable, can be employed. Thus, it might be inferred that the entity did want the action performed.

The entity can also interact with the regions to select and provide information through various devices such as a mouse, a roller ball, a keypad, a keyboard, a pen, gestures captured with a camera, a touch screen, and/or voice activation, for example. According to an aspect, a mechanism, such as a push button or the enter key on the keyboard, can be employed subsequent to entering the information in order to initiate information conveyance. However, it is to be appreciated that the disclosed aspects are not so limited. For example, merely highlighting a check box can initiate information conveyance. In another example, a command line interface can be employed. For example, the command line interface can prompt the entity for information by providing a text message, producing an audio tone, or the like. The entity can then provide suitable information, such as alphanumeric input corresponding to an option provided in the interface prompt or an answer to a question posed in the prompt. It is to be appreciated that the command line interface can be employed in connection with a GUI and/or Application Program Interface (API). In addition, the command line interface can be employed in connection with hardware (e.g., video cards) and/or displays (e.g., black and white, and Video Graphics Array (VGA)) with limited graphic support, and/or low bandwidth communication channels.

The at least one memory 918 can be operatively connected to the at least one processor 920. The at least one memory 918 can store executable instructions and/or computer executable components (e.g., the extraction component 902, the reduction component 904, the locator component 906, the scoring component 908, the classification component 910, the execution component 912, the user interface component 914, the transmitter/receiver component 916, and so on) that, when executed by the at least one processor 920, can facilitate performance of operations (e.g., the operations discussed with respect to the various methods and/or systems discussed herein). Further, the at least one processor 920 can be utilized to execute computer executable components (e.g., the extraction component 902, the reduction component 904, the locator component 906, the scoring component 908, the classification component 910, the execution component 912, the user interface component 914, the transmitter/receiver component 916, and so on) stored in the at least one memory 918.

For example, the at least one memory 918 can store protocols associated with facilitating identifying peptides with multiple post-translational modification using a greedy approach as discussed herein. Further, the at least one memory 918 can facilitate action to control communication between the system 900 and other systems, user equipment, one or more file storage systems, one or more devices, such that the system 900 employ stored protocols and/or processes to achieve improved overall performance using large language models as described herein.

It should be appreciated that data stores (e.g., memories) components described herein can be either volatile memory or nonvolatile memory, or can include both volatile and nonvolatile memory. By way of example and not limitation, nonvolatile memory can include read only memory (ROM), programmable ROM (PROM), electrically programmable ROM

(EPROM), electrically erasable ROM (EEPROM), or flash memory. Volatile memory can include random access memory (RAM), which acts as external cache memory. By way of example and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Memory of the disclosed aspects are intended to comprise, without being limited to, these and other suitable types of memory.

The at least one processor 920 can facilitate respective analysis of information related to identifying peptides with multiple post-translational modification as discussed herein. The at least one processor 920 can be a processor dedicated to analyzing and/or generating information received, a processor that controls one or more components of the system 900, and/or a processor that both analyzes and generates information received and controls one or more components of the system 900.

The transmitter/receiver component 916 can receive one or more commands and/or response as discussed herein. The transmitter/receiver component 916 can be configured to transmit to, and/or receive data from, for example, a user equipment, a client, network equipment, the extraction component 902, the reduction component 904, the locator component 906, the scoring component 908, the classification component 910, the execution component 912, the user interface component 914, the transmitter/receiver component 916, and/or other communication component and/or devices. Through the transmitter/receiver component 916, the system 900 can concurrently transmit and receive data, can transmit and receive data at different times, or combinations thereof.

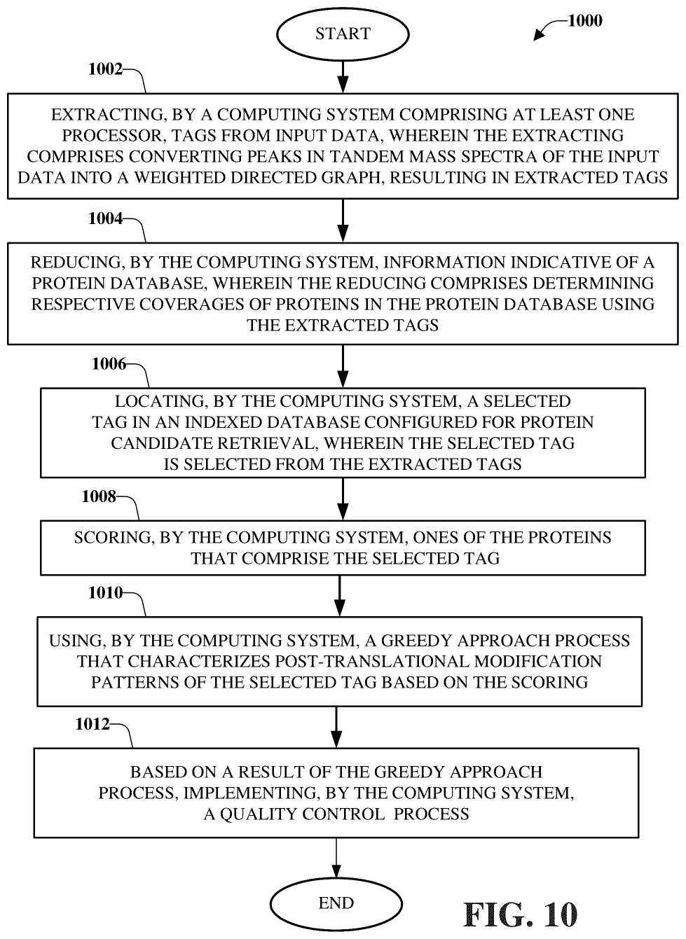

FIG. 10 illustrates an example, non-limiting, computer-implemented method 1000 that facilitates identification of peptides with multiple post-translational modification in accordance with one or more embodiments described herein. The computer-implemented method 1000 and/or other methods discussed herein can be implemented by network equipment comprising a processor. According to another example, the computer-implemented method can be implemented by a system (e.g., the system workflow of FIGS. 1A-1C, the system 900, and so on) comprising at least one processor and at least one memory.

The computer-implemented method 1000 starts, at 1002, with extracting, by a computing system comprising at least one processor, tags from input data. The extracting can include converting peaks in tandem mass spectra of the input data into a weighted directed graph, resulting in extracted tags. The tags represent sequential amino acids. The tandem mass spectra can be obtained (prior to the extracting) from a group of proteins with post-translational modifications. In some implementations, the extracting can include extracting the tags using a depth-first search process.

At 1004, the computer-implemented method 1000 reduces, by the computing system, information indicative of a protein database. The reducing can include determining respective coverages of proteins in the protein database using the extracted tags. In an implementation, the reducing can include removing any of the proteins determined to have a coverage level that is below a threshold coverage level.

The computer-implemented method, at 1006, locates, by the computing system, a selected tag in an indexed database configured for protein candidate retrieval. The selected tag is selected from the extracted tags. In an implementation, the locating at 1006 includes using tags comprising ammino acid lengths between 3 amino acids and 9 amino acids for the retrieval of protein candidates.

According to an implementation, prior to the locating at 1006, the computer-implemented method 1000 constructs, by the computing system, the indexed database that facilitates protein candidate retrieval using tags of various lengths. Further to these implementations, the constructing comprises, based on a determination that the quality control process has been applied, generating a target indexed database and a decoy indexed database. In an example, generating of the decoy indexed database can include shuffling target protein sequences.

Further, at 1008, the computer-implemented method 1000 can facilitate scoring, by the computing system, ones of the proteins that comprise the selected tag. For example, proteins that comprise the selected tag are scored while other proteins that do not comprise the selected tag are not scored.

A greedy approach process is used, at 1010, for characterizing post-translational modification patterns of the selected tag based on the scoring. Based on a result of the greedy approach process, at 1012, a quality control process is implemented. In an example, using of the greedy approach process can include iteratively including a current best post-translational modification pattern of the post-translational modification patterns with which a largest number of experimental peaks are matched to theoretical peaks. The experimental peaks and the theoretical peaks are generated from protein candidates in the indexed database.

According to some implementations, the tags include respective N-sections and respective C-sections. Therefore, using of the greedy approach process, at 1010, can include using the greedy approach process on the respective N-sections and the respective C-sections.

According to some implementations, the computer-implemented method 1000 facilitates, by the computing system, digestion of samples of the group of proteins into peptides by an enzyme before using the tandem mass spectra. Further to these implementations, the computer-implemented method 1000 transmits, by the computing system, the samples with the post-translational modifications to a tandem mass spectrometer.

The computer-implemented method 1000 can include, according to some implementations, determining, by the computing system, nodes and edges of the weighted directed graph that generates tags. The determining of the nodes and the edges can include using peaks in the MS2 spectra and potential amino acids that are located between peak pairs of the tags.

Provided herein are embodiments that targets identifying peptides with unspecified multiple PTMs with a greedy approach. In contrast, conventional methods can only identify pre-specified PTMs, and the number is limited because of algorithm complexity. Further, the embodiments provided herein use tag-based strategy to detect backbone sequences of the modified peptides. Tags are invariant to PTMs, whereas at least one conventional method uses an ion-based method that cannot benefit from tags.

Additionally, the embodiments provided herein identify multiple PTMs on one peptide using a greedy approach, while conventional methods identify multiple PTMs by enumerating different PTM combinations. The performances of the conventional methods are limited by the exponentially increasing number of PTM combinations when considering more PTM candidates. Also, conventional methods use fixed lengths, ignoring the effect of different tag lengths in the identification of peptides. In contrast, the disclosed embodiments combine tags of various lengths, which is a simple and effective solution to the problem of choosing a suitable tag length for retrieving peptide candidates in data sets of different complexity.

Advantages of the disclosed embodiments include, but are not limited to being the first method that uses a greedy approach to identifying peptides with multiple PTMs. The analysis tool provided herein was developed using JAVA programming language to identify peptides with multiple PTMs by iteratively including the current best single PTMs to form a final PTM pattern. This strategy bypasses the exponential complexity of the huge number of PTM combinations. The disclosed embodiments can achieve significantly better performance on peptides with multiple PTMs than conventional methods. Besides, the disclosed embodiments provide a method that can maintain its performance when the data quality decreases. Additionally, the disclosed embodiments combine tags of various lengths to further improve tag-based peptide identification methods. The disclosed embodiments can also benefit other tag-based methods.

The various embodiments (PIPI2) provided herein can serve as an effective and efficient analysis tool to identify peptides with multiple PTMs. It can provide a service to at least the following markets: Proteomics Research Tools, Biomedical Applications, Research and Academic Institutions, and/or Bioinformatics and Software Development Companies.

Proteomics Research Tools: PIPI2 can be utilized as a valuable tool for researchers and scientists involved in proteomics research. It offers enhanced peptide identification and PTM characterization capabilities, particularly for peptides with multiple PTMs. The embodiments may enable the development of software packages, algorithms, or databases specifically tailored for proteomics analysis.

Biomedical Applications: The accurate identification and characterization of peptides with PTMs has significant implications in understanding cellular activities and regulatory mechanisms. PIPI2's improved performance in handling peptides with multiple PTMs can contribute to advancements in biomedical research, drug discovery, and personalized medicine.

Research and Academic Institutions: Universities, research institutes, and biomedical laboratories involved in proteomics research are potential users of PIPI2. The disclosed embodiments can enhance their research capabilities and contribute to scientific discoveries.

Bioinformatics and Software Development Companies: Organizations specializing in bioinformatics, software development, and computational biology may incorporate PIPI2's algorithms and methodologies into their existing proteomics software suites, for example.

Additional advantages of the disclosed embodiments include, but are not limited to: Tag-Based Strategy, Greedy Approach for Multiple PTMs, and Variable Tag Lengths.

Tag-Based Strategy: Unlike conventional methods that use ion-based methods, the disclosed embodiments employ a tag-based strategy for detecting the backbone sequences of modified peptides. Tags are invariant to PTMs, which allows for more accurate identification of peptides regardless of the specific modifications present. This approach provides a distinct advantage over conventional methods that cannot benefit from tags in their analysis.

Greedy Approach for Multiple PTMs: In contrast to conventional methods that enumerate different PTM combinations to identify multiple PTMs on a peptide, the disclosed embodiments utilize a greedy approach. By simplifying the combinatorial problem to a linear one, the disclosed embodiments can handle peptides with multiple PTMs more efficiently. This approach overcomes the limitations faced by conventional methods, as their performance is hindered by the exponentially increasing number of PTM combinations when considering more PTM candidates.

Variable Tag Lengths: The disclosed embodiments combines tags of various lengths and addresses the problem of choosing a suitable tag length for retrieving peptide candidates in datasets of different complexity. Unlike conventional methods that use fixed lengths and ignore the effect of different tag lengths, the disclosed embodiments provide a simple and effective solution, enhancing the accuracy and flexibility of peptide identification.

Methods that can be implemented in accordance with the disclosed subject matter will be better appreciated with reference to the flow charts provided herein. While, for purposes of simplicity of explanation, the methods are shown and described as a series of flows and/or blocks, it is to be understood and appreciated that the disclosed aspects are not limited by the number or order of flows and/or blocks, as some flows and/or blocks can occur in different orders and/or at substantially the same time with other blocks from what is depicted and described herein. Moreover, not all illustrated flows and/or blocks are required to implement the disclosed methods. It is to be appreciated that the functionality associated with the flows and/or blocks can be implemented by software, hardware, a combination thereof, or any other suitable means (e.g., device, system, process, component, and so forth). Additionally, it should be further appreciated that the disclosed methods are capable of being stored on an article of manufacture to facilitate transporting and transferring such methods to various devices. Those skilled in the art will understand and appreciate that the methods could alternatively be represented as a series of interrelated states or events, such as in a state diagram.

Aspects of systems, devices, apparatuses, and/or processes explained in this disclosure can constitute machine-executable component(s) embodied within machine(s) (e.g., embodied in one or more computer readable mediums (or media) associated with one or more machines). Such component(s), when executed by the one or more machines (e.g., computer(s), computing device(s), virtual machine(s), and so on) can cause the machine(s) to perform the operations described.

In various embodiments, the system can be any type of component, machine, device, facility, apparatus, and/or instrument that comprises a processor and/or can be capable of effective and/or operative communication with a wired and/or wireless network. Components, machines, apparatuses, devices, facilities, and/or instrumentalities that can comprise the system can include tablet computing devices, handheld devices, server class computing machines and/or databases, laptop computers, notebook computers, desktop computers, cell phones, smart phones, consumer appliances and/or instrumentation, industrial and/or commercial devices, hand-held devices, digital assistants, multimedia Internet enabled phones, multimedia players, and the like.

As used herein, the term “storage device,” “first storage device,” “second storage device,” “storage cluster nodes,” “storage system,” “data store” and the like (e.g., node device), can include, for example, private or public cloud computing systems for storing data as well as systems for storing data comprising virtual infrastructure and those not comprising virtual infrastructure. The term “I/O request” (or simply “I/O”) can refer to a request to read and/or write data.

The term “cloud” as used herein can refer to a cluster of nodes (e.g., set of network servers), for example, within an object storage system, which are communicatively and/or operatively coupled to one another, and that host a set of applications utilized for servicing user requests. In general, the cloud computing resources can communicate with user devices via most any wired and/or wireless communication network to provide access to services that are based in the cloud and not stored locally (e.g., on the user device). A typical cloud-computing environment can include multiple layers, aggregated together, that interact with one another to provide resources for end-users.

Further, the term “storage device” can refer to any Non-Volatile Memory (NVM) device, including Hard Disk Drives (HDDs), flash devices (e.g., NAND flash devices), and next generation NVM devices, any of which can be accessed locally and/or remotely (e.g., via a Storage Attached Network (SAN)). In some embodiments, the term “storage device” can also refer to a storage array comprising one or more storage devices. In various embodiments, the term “object” refers to an arbitrary-sized collection of user data that can be stored across one or more storage devices and accessed using I/O requests.

Further, a storage cluster can include one or more storage devices. For example, a storage system can include one or more clients in communication with a storage cluster via a network. The network can include various types of communication networks or combinations thereof including, but not limited to, networks using protocols such as Ethernet, Internet Small Computer System Interface (iSCSI), Fibre Channel (FC), and/or wireless protocols. The clients can include user applications, application servers, data management tools, and/or testing systems.

As utilized herein an “entity,” “client,” “user,” and/or “application” can refer to any system or person that can send I/O requests to a storage system. For example, an entity, can be one or more computers, the Internet, one or more systems, one or more commercial enterprises, one or more computers, one or more computer programs, one or more machines, machinery, one or more actors, one or more users, one or more customers, one or more humans, and so forth, hereinafter referred to as an entity or entities depending on the context.

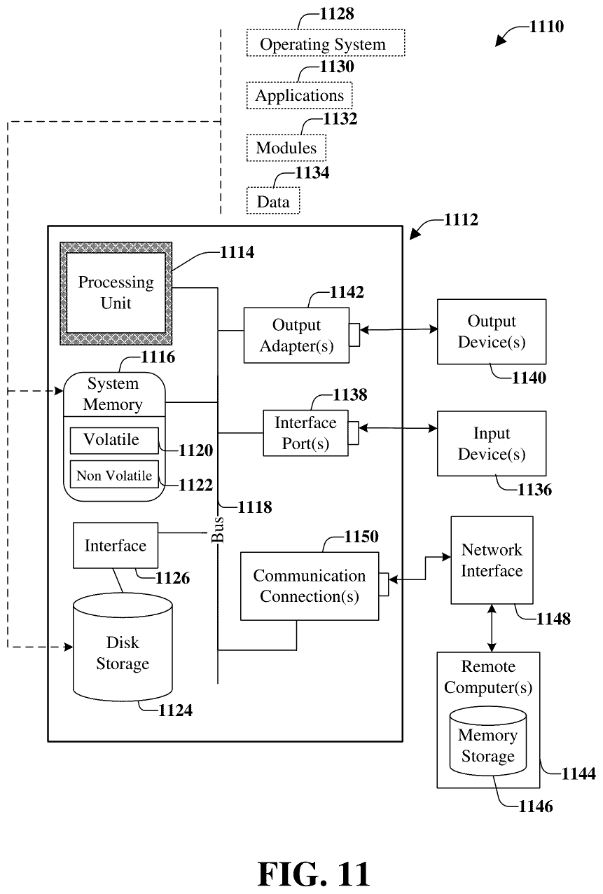

In order to provide a context for the various aspects of the disclosed subject matter, FIG. 11 as well as the following discussion are intended to provide a brief, general description of a suitable environment in which the various aspects of the disclosed subject matter can be implemented.

With reference to FIG. 11, an example environment 1110 for implementing various aspects of the aforementioned subject matter comprises a computer 1112. The computer 1112 comprises a processing unit 1114, a system memory 1116, and a system bus 1118. The system bus 1118 couples system components including, but not limited to, the system memory 1116 to the processing unit 1114. The processing unit 1114 can be any of various available processors. Multi-core microprocessors and other multiprocessor architectures also can be employed as the processing unit 1114.

The system bus 1118 can be any of several types of bus structure(s) including the memory bus or memory controller, a peripheral bus or external bus, and/or a local bus using any variety of available bus architectures including, but not limited to, 8-bit bus, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Universal Serial Bus (USB), Advanced Graphics Port (AGP), Personal Computer Memory Card International Association bus (PCMCIA), and Small Computer Systems Interface (SCSI).

The system memory 1116 comprises volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can comprise read only memory (ROM), programmable ROM (PROM), electrically programmable ROM (EPROM), electrically crasable PROM (EEPROM), or flash memory.

Volatile memory 1120 comprises random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM).

Computer 1112 also comprises removable/non-removable, volatile/non-volatile computer storage media. FIG. 11 illustrates, for example, a disk storage 1124. Disk storage 1124 comprises, but is not limited to, devices like a magnetic disk drive, floppy disk drive, tape drive, Jaz drive, Zip drive, LS-100 drive, flash memory card, or memory stick. In addition, disk storage 1124 can comprise storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1124 to the system bus 1118, a removable or non-removable interface is typically used such as interface 1126.

According to some implementations, the example environment 1110 can include non-transitory machine-readable medium, comprising executable instructions that, when executed by at least one processor of network equipment, facilitate performance of operations. The operations can include retrieving peptide backbone candidates using tags of various lengths. The peptide backbone candidates can include multiple post-translational modification patterns, and wherein the tags represent sequential amino acids. The operations can also include characterizing post-translational modification patterns of the multiple post-translational modification patterns of the peptide backbone candidates by employing a greedy approach that simplifies a combinatorial problem into a linear problem, resulting in characterized candidates. Further, the operations can include scoring the characterized candidates in an indexed database, resulting in scored candidates. The operations can also include applying a protein feedback process that re-ranks the scored candidates based on respective scores of proteins that contain the characterized candidates, resulting in re-ranked candidates. In addition, the operations can include outputting the re-ranked candidates while concurrently controlling a false discovery rate with a quality control process.

According to some implementations, to characterize the post-translational modification patterns, the operations can include identifying peptides with multiple post-translational modifications without user specification. In an example, the quality control process can facilitate estimation of an output quality applicable to the re-ranked candidates.

It is to be appreciated that FIG. 11 describes software that acts as an intermediary between users and the basic computer resources described in suitable operating environment 1110. Such software comprises an operating system 1128. Operating system 1128, which can be stored on disk storage 1124, acts to control and allocate resources of the computer 1112. System applications 1130 take advantage of the management of resources by operating system 1128 through program modules 1132 and program data 1134 stored either in system memory 1116 or on disk storage 1124. It is to be appreciated that one or more embodiments of the subject disclosure can be implemented with various operating systems or combinations of operating systems.

A user enters commands or information into the computer 1112 through input device(s) 1136. Input devices 1136 comprise, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 comprise, for example, a serial port, a parallel port, a game port, and a universal serial bus (USB). Output device(s) 1140 use some of the same type of ports as input device(s) 1136. Thus, for example, a USB port can be used to provide input to computer 1112, and to output information from computer 1112 to an output device 1140. Output adapters 1142 are provided to illustrate that there are some output devices 1140 like monitors, speakers, and printers, among other output devices 1140, which require special adapters. The output adapters 1142 comprise, by way of illustration and not limitation, video and sound cards that provide a means of connection between the output device 1140 and the system bus 1118. It should be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a personal computer, a server, a router, a network PC, a workstation, a microprocessor based appliance, a peer device or other common network node and the like, and typically comprises many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses communication networks such as local-area networks (LAN) and wide-area networks (WAN). LAN technologies comprise Fiber Distributed Data Interface (FDDI), Copper Distributed Data Interface (CDDI), Ethernet/IEEE 802.3, Token Ring/IEEE 802.5, and the like. WAN technologies comprise, but are not limited to, point-to-point links, circuit switching networks like Integrated Services Digital Networks (ISDN) and variations thereon, packet switching networks, and Digital Subscriber Lines (DSL).

Communication connection(s) 1150 refers to the hardware/software employed to connect the network interface 1148 to the system bus 1118. While communication connection 1150 is shown for illustrative clarity inside computer 1112, it can also be external to computer 1112. The hardware/software necessary for connection to the network interface 1148 comprises, for exemplary purposes only, internal, and external technologies such as, modems including regular telephone grade modems, cable modems and DSL modems, ISDN adapters, and Ethernet cards.

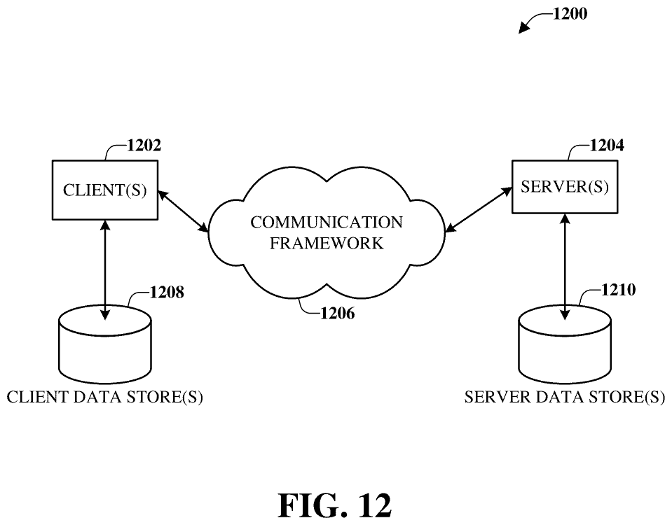

FIG. 12 is a schematic block diagram of a sample computing environment 1200 with which the disclosed subject matter can interact. The sample computing environment 1200 includes one or more client(s) 1202. The client(s) 1202 can be hardware and/or software (e.g., threads, processes, computing devices). The sample computing environment 1200 also includes one or more server(s) 1204. The server(s) 1204 can also be hardware and/or software (e.g., threads, processes, computing devices). The servers 1204 can house threads to perform transformations by employing one or more embodiments as described herein, for example. One possible communication between a client 1202 and servers 1204 can be in the form of a data packet adapted to be transmitted between two or more computer processes. The sample computing environment 1200 includes a communication framework 1206 that can be employed to facilitate communications between the client(s) 1202 and the server(s) 1204. The client(s) 1202 are operably connected to one or more client data store(s) 1208 that can be employed to store information local to the client(s) 1202. Similarly, the server(s) 1204 are operably connected to one or more server data store(s) 1210 that can be employed to store information local to the servers 1204.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics can be combined in any suitable manner in one or more embodiments.

As used in this disclosure, in some embodiments, the terms “component,” “system,” “interface,” “manager,” and the like are intended to refer to, or comprise, a computer-related entity or an entity related to an operational apparatus with one or more specific functionalities, wherein the entity can be either hardware, a combination of hardware and software, software, or software in execution, and/or firmware. As an example, a component can be, but is not limited to being, a process running on a processor, a processor, an object, an executable, a thread of execution, computer-executable instructions, a program, and/or a computer. By way of illustration and not limitation, both an application running on a server and the server can be a component.

One or more components can reside within a process and/or thread of execution and a component can be localized on one computer and/or distributed between two or more computers. In addition, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by one or more processors, wherein the processor can be internal or external to the apparatus and can execute at least a part of the software or firmware application. Yet another example, a component can be an apparatus that provides specific functionality through electronic components without mechanical parts, the electronic components can comprise a processor therein to execute software or firmware that confer(s), at least in part, the functionality of the electronic components. In an aspect, a component can emulate an electronic component via a virtual machine, e.g., within a cloud computing system. While various components have been illustrated as separate components, it will be appreciated that multiple components can be implemented as a single component, or a single component can be implemented as multiple components, without departing from example embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, the various embodiments can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, machine-readable device, computer-readable carrier, computer-readable media, machine-readable media, computer-readable (or machine-readable) storage/communication media. For example, computer-readable storage media can comprise, but are not limited to, random access memory (RAM), read only memory (ROM), electrically erasable programmable read only memory (EEPROM), flash memory or other memory technology, solid state drive (SSD) or other solid-state storage technology, a magnetic storage device, e.g., hard disk; floppy disk; magnetic strip(s); an optical disk (e.g., compact disk (CD), a digital video disc (DVD), a Blu-ray Disc™ (BD)); a smart card; a flash memory device (e.g., card, stick, key drive); and/or a virtual device that emulates a storage device and/or any of the above computer-readable media. Of course, those skilled in the art will recognize that many modifications can be made to this configuration without departing from the scope or spirit of the various embodiments.

Disclosed embodiments and/or aspects should neither be presumed to be exclusive of other disclosed embodiments and/or aspects, nor should a device and/or structure be presumed to be exclusive to its depicted element in an example embodiment or embodiments of this disclosure, unless where clear from context to the contrary. The scope of the disclosure is generally intended to encompass modifications of depicted embodiments with additions from other depicted embodiments, where suitable, interoperability among or between depicted embodiments, where suitable, as well as addition of a component(s) from one embodiment(s) within another or subtraction of a component(s) from any depicted embodiment, where suitable, aggregation of elements (or embodiments) into a single device achieving aggregate functionality, where suitable, or distribution of functionality of a single device into multiple device, where suitable. In addition, incorporation, combination or modification of devices or elements (e.g., components) depicted herein or modified as stated above with devices, structures, or subsets thereof not explicitly depicted herein but known in the art or made evident to one with ordinary skill in the art through the context disclosed herein are also considered within the scope of the present disclosure.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGS., where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A method, comprising:
extracting, by a computing system comprising at least one processor, tags from input data, wherein the extracting comprises converting peaks in tandem mass spectra of the input data into a weighted directed graph, resulting in extracted tags, wherein the tags represent sequential amino acids;
reducing, by the computing system, information indicative of a protein database, wherein the reducing comprises determining respective coverages of proteins in the protein database using the extracted tags;
locating, by the computing system, a selected tag in an indexed database configured for protein candidate retrieval, wherein the selected tag is selected from the extracted tags;
scoring, by the computing system, ones of the proteins that comprise the selected tag;
using, by the computing system, a greedy approach process that characterizes post-translational modification patterns of the selected tag based on the scoring; and
based on a result of the greedy approach process, implementing, by the computing system, a quality control process.

2. The method of claim 1, wherein the tags comprise respective N-sections and respective C-sections, and wherein the using of the greedy approach process comprises using the greedy approach process on the respective N-sections and the respective C-sections.

3. The method of claim 1, further comprising:
prior to the extracting, obtaining, by the computing system, the tandem mass spectra from a group of proteins with post-translational modifications.

4. The method of claim 3, further comprising:
facilitating, by the computing system, digestion of samples of the group of proteins into peptides by an enzyme before using the tandem mass spectra.

5. The method of claim 4, further comprising:
transmitting, by the computing system, the samples with the post-translational modifications to a tandem mass spectrometer.

6. The method of claim 1, further comprising:
determining, by the computing system, nodes and edges of a weighted directed graph utilized to extract the tags, wherein the determining of the nodes and the edges comprises using peaks in the tandem mass spectra and potential amino acids that are located between peak pairs of the tags.

7. The method of claim 1, wherein the extracting comprises extracting the tags using a depth-first search process.

8. The method of claim 1, wherein the reducing comprises removing any of the proteins determined to have a coverage level that is below a threshold coverage level.

9. The method of claim 1, further comprising:
prior to the locating, constructing, by the computing system, the indexed database that facilitates protein candidate retrieval using tags of various lengths.

10. The method of claim 9, wherein the constructing comprises, based on a determination that the quality control process has been applied, generating a target indexed database and a decoy indexed database.

11. The method of claim 10, wherein the generating of the decoy indexed database comprises shuffling target protein sequences.

12. The method of claim 1, wherein the locating comprises using tags comprising ammino acid lengths between 3 amino acids and 9 amino acids for retrieval of protein candidates.

13. The method of claim 1, wherein the using of the greedy approach process comprises iteratively including a current best post-translational modification pattern of the post-translational modification patterns with which a largest number of experimental peaks are matched to theoretical peaks, wherein the experimental peaks are generated from the tandem mass spectra and the theoretical peaks are generated from protein candidates in the indexed database.

14. A system, comprising:
at least one processor; and
at least one memory that stores executable instructions that, when executed by the at least one processor, facilitate performance of operations, comprising:
extracting tags that represent sequential amino acids based on peaks in tandem mass spectra being converted into a weighted directed graph, resulting in extracted tags;
reducing a protein database based on the extracted tags, wherein the reducing comprises removing, from the protein database, extracted tags determined to have a reliability level that is below a reliability threshold level, wherein the reliability level is based on a determination of respective coverages of proteins in the protein database;
locating a tag in an indexed database, resulting in a located tag, wherein the extracted tags comprise the located tag;
scoring ones of the proteins that comprise the located tag;
based on the scoring, characterizing post-translational modification patterns of the located tag, wherein the characterizing comprises using a greedy approach process; and
based on a result of the greedy approach process, implementing a quality control process.

15. The system of claim 14, wherein the operations further comprise:
prior to the extracting, obtaining the tandem mass spectra from a group of proteins with post-translational modifications.

16. The system of claim 14, wherein the extracted tags comprises tags of different lengths, and wherein the identifying is performed without user specification.

17. The system of claim 14, wherein the operations further comprise:
prior to the identifying, generating the indexed database, wherein the indexed database facilitates protein candidate retrieval using tags of different lengths.

18. A computing system, comprising at least one processor configured to:
retrieve peptide backbone candidates using tags of various lengths, wherein the peptide backbone candidates comprise multiple post-translational modification patterns, and wherein the tags represent sequential amino acids;
characterize post-translational modification patterns of the multiple post-translational modification patterns of the peptide backbone candidates by employing a greedy approach that simplifies a combinatorial problem into a linear problem, resulting in characterized candidates;
score the characterized candidates in an indexed database, resulting in scored candidates;
apply a protein feedback process that re-ranks the scored candidates based on respective scores of proteins that contain the characterized candidates, resulting in re-ranked candidates; and
output the re-ranked candidates while concurrently controlling a false discovery rate with a quality control process.

19. The computing system of claim 18, wherein, to characterize the post-translational modification patterns, the at least one processor is configured to identify peptides with multiple post-translational modifications in absence of any user specification.

20. The computing system of claim 18, wherein the quality control process facilitates estimation of an output quality applicable to the re-ranked candidates.
